(19) United States
(12) Patent Application Publication
(10) Pub. No.: US 2021/0340173 A1
(43) Pub. Date: Nov. 4, 2021

(54) METHODS FOR PRODUCING A PLURALITY OF POLYPEPTIDE VARIANTS SUITABLE FOR BIOLOGICAL ANALYSIS

(71) Applicant: UNIVERSITÉ DE GENÈVE, Geneva (CH)

(72) Inventors: Oliver Hartley, Carouge (CH); Marianne Paolini-Bertrand, Habere-Poche (FR)

(73) Assignee: UNIVERSITÉ DE GENÈVE, Geneva (CH)

(21) Appl. No.: 17/282,220

(22) PCT Filed: Sep. 25, 2019
(86) PCT No.: PCT/IB2019/058129

§ 371 Date: Apr. 1, 2021

Related U.S. Application Data
(60) Provisional application No. 62/739,555, filed on Oct. 1, 2018.

(51) Int. Cl. C07K 1/02; G01N 33/50

(52) U.S. Cl. CPC C07K 1/026; G01N 33/5008

(57) ABSTRACT

The present application relates to methods for producing structurally variant polypeptide molecules in parallel using column-free techniques by polypeptide ligation, separation of ligated polypeptides from ligation reactions, folding of polypeptides, and desalting of polypeptides. Further described are methods for determining the effects and properties of structurally variant polypeptide molecules produced in parallel using column-free techniques.

### CROSS-REFERENCE TO RELATED APPLICATIONS

This application claims the benefit of and priority from U.S. Provisional Patent Application No. 62/739,555 filed on Oct. 1, 2018 which is hereby incorporated by reference in its entirety.

### FIELD

The present application relates to methods for producing folded structurally variant polypeptide molecules by polypeptide ligation and folding, and analyzing said folded structurally variant polypeptide molecules to determine their effects and properties.

### BACKGROUND

In the field of biological research, and in pharmacological research in particular, it is important to identify molecules that have desirable effects. Desirable biological effects include, without limitation, binding to a ligand or receptor, blocking a ligand or receptor, causing a receptor to be internalized within the cell, selectively activating receptor signalling pathways, stimulating cells, killing cells, and modulating cells. Desirable medical effects of a molecule include, without limitation, killing bacteria, disabling viruses, killing cancer cells, inhibiting cell proliferation, inhibiting disease pathways, and restoring the function of healthy pathways.

Once a candidate molecule has been identified as having one or more desirable effects, it is further possible to improve the effects or the properties of the candidate molecule by creating variants of that candidate molecule. It is possible that variants of a candidate molecule will produce greater magnitudes of desired effects or reduced magnitudes of undesired effects. As well, variants of the candidate molecule may also exhibit enhanced properties including improved stability, improved solubility, reduced toxicity, and increased or decreased binding to specific ligands or receptors.

Often it is unknown which specific variants, or even which general category of variants, of a candidate molecule will have improved properties. As well, it is possible that some variants will possess unforeseen and surprising properties. Thus, it is useful to generate large numbers of variants and screen them all for their effects and properties. Furthermore, it is efficient and economical to generate a large number of variants in parallel (i.e. a molecular library) and then to screen the variants for their effects and properties in parallel. Such large scale, parallel screening allows for the rapid identification of useful variants among the larger number of non-useful variants. Useful variants identified in this manner can then be selected for further investigation.

Polypeptides are an important class of molecule for biological and medical research. Polypeptides are polymers of amino acids and are the primary constituent of proteins. For the production of smaller peptides up to 25 amino acid residues in length, existing technologies can readily be used for the parallel production and screening of large peptide libraries. However, these technologies are not suitable for longer polypeptides, including the polypeptides that constitute proteins. The reliability of peptide synthesis decreases sharply after 25 residues. Furthermore, the synthesis of longer polypeptides requires time-consuming and costly purification steps such as column chromatography. Column chromatography is laborious, time-consuming, and costly and is therefore not amenable to the parallel production and screening of multiple polypeptide variants.

Producing polypeptides by recombinant expression or phage display requires extensive cloning, subcloning, expression, and purification steps that significantly limit the ability to screen molecules quickly and in parallel in large numbers.

Other techniques to generate polypeptide variants employ the synthesis of shorter peptide fragments followed by the chemical ligation of the fragments to produce longer polypeptides. Though effective for the production of a small selection of polypeptide variants, these techniques require one or more column chromatography steps for the purification of the polypeptide (Canne U.S. Pat. No. 7,094,871; Low WO2004105685). As stated above, column chromatography is not amenable to the parallel production and screening of multiple polypeptide variants.

Techniques of peptide ligation have been developed that do not require column chromatography of the ligated polypeptide (Loibl 2016). However, these techniques require the addition of covalently-linked tags onto the ends of the peptide fragments, and such tags can affect the properties of the final polypeptide molecule. Furthermore, these techniques require the multiple tag-based resin-capture steps, thereby adding cost and complexity. These features inhibit the scale and applicability of such techniques for parallel production and screening.

Therefore, a need exists for a method of producing large numbers of polypeptide variants without limiting features such as in vivo expression, column chromatography, or capturing tagged peptides on a solid-phase. Because such features restrict the ability to produce and screen large numbers of polypeptide variants in parallel, a method without these features would have great utility by reducing the time and cost required to identify polypeptide variants with properties useful for research and medicine. Such a method would also need to produce the polypeptide variants in such a state that their biological effects and properties are preserved and suitable for analysis.

The present application discloses a novel method for producing large numbers of polypeptide variants in parallel and in a form that is suitable for the analysis of their effects and properties. Importantly, the method of the present application does not require the modification of the polypeptides with tags, nor does it require that the polypeptide variants be purified by column chromatography. The method of the present invention therefore allows for the production of large numbers of polypeptide variants in parallel and in a form suitable for analysis, and the subsequent screening of said polypeptide variants for useful effects and properties.

### SUMMARY

In an embodiment, the present invention relates to a method for producing a large number of polypeptide variants in parallel and in a form suitable for screening and analysis in parallel.

In another embodiment, the present invention relates to a method for producing a large number of polypeptide variants in parallel and in a form suitable for determining at least one effect and/or at least one property of said polypeptide variants.

In another embodiment, the present invention relates to a method for producing a plurality of structurally variant polypeptide molecules in parallel comprising: a) providing a plurality of structurally variant regions of a polypeptide molecule in parallel, b) ligating each of said plurality of structurally variant regions of a polypeptide molecule to a common, structurally invariant region of said polypeptide molecule in parallel separate ligation reactions to produce a plurality of structurally variant polypeptide molecules, and c) applying conditions to each of said separate ligation reactions in parallel to separate said plurality of structurally variant polypeptide molecules from each of said separate ligation reactions.

In another embodiment, the present invention relates to a method for producing a plurality of structurally variant polypeptide molecules in parallel comprising: a) providing a plurality of structurally variant regions of a polypeptide molecule in parallel, b) ligating each of said plurality of structurally variant regions of a polypeptide molecule to a common, structurally invariant region of said polypeptide molecule in parallel separate ligation reactions to produce a plurality of structurally variant polypeptide molecules, and c) applying conditions to each of said separate ligation reactions in parallel to separate said plurality of structurally variant polypeptide molecules from each of said separate ligation reactions, and d) lyophilizing each of said plurality of folded structurally variant polypeptide molecules in parallel.

In another embodiment, the present invention relates to a method for producing a plurality of folded structurally variant polypeptide molecules in parallel comprising: a) providing a plurality of structurally variant regions of a polypeptide molecule in parallel, b) ligating each of said plurality of structurally variant regions of a polypeptide molecule to a common, structurally invariant region of said polypeptide molecule in parallel separate ligation reactions to produce a plurality of structurally variant polypeptide molecules, c) applying conditions to each of said separate ligation reactions in parallel to separate said plurality of structurally variant polypeptide molecules from each of said separate ligation reactions, d) folding each of said plurality of structurally variant polypeptide molecules in parallel separate folding reactions to produce a plurality of folded structurally variant polypeptide molecules, and e) applying conditions to each of said separate folding reactions in parallel to separate said plurality of folded structurally variant polypeptide molecules from said folding reactions.

In another embodiment, the present invention relates to a method for producing a plurality of folded structurally variant polypeptide molecules in parallel comprising: a) providing a plurality of structurally variant regions of a polypeptide molecule in parallel, b) ligating each of said plurality of structurally variant regions of a polypeptide molecule to a common, structurally invariant region of said polypeptide molecule in parallel separate ligation reactions to produce a plurality of structurally variant polypeptide molecules, c) applying conditions to each of said separate ligation reactions in parallel to separate said plurality of structurally variant polypeptide molecules from each of said separate ligation reactions, d) folding each of said plurality of structurally variant polypeptide molecules in parallel separate folding reactions to produce a plurality of folded structurally variant polypeptide molecules, e) applying conditions to each of said separate folding reactions in parallel to separate said plurality of folded structurally variant polypeptide molecules from said folding reactions, and f) lyophilizing each of said plurality of folded structurally variant polypeptide molecules in parallel.

In another embodiment, the present invention relates to a method for determining at least one effect of a plurality of structurally variant polypeptide molecules in parallel comprising: a) providing a plurality of structurally variant polypeptide molecules by the method as described herein, b) contacting the plurality of structurally variant polypeptide molecules separately in parallel with cells, and c) determining at least one effect of the plurality of structurally variant polypeptide molecules on said cells.

In another embodiment, the present invention relates to a method for determining at least one effect of a plurality of folded structurally variant polypeptide molecules in parallel comprising: a) providing a plurality of folded structurally variant polypeptide molecules by the method as described herein, b) contacting the plurality of folded structurally variant polypeptide molecules separately in parallel with cells, and c) determining at least one effect of the plurality of folded structurally variant polypeptide molecules on said cells.

In another embodiment, the present invention relates to a method for determining at least one property of a plurality of structurally variant polypeptide molecules in parallel comprising: a) providing a plurality of structurally variant polypeptide molecules produced by the method as described herein, and b) determining at least one property of the plurality of structurally variant polypeptide molecules.

In another embodiment, the present invention relates to a method for determining at least one property of a plurality of folded structurally variant polypeptide molecules in parallel comprising: a) providing a plurality of folded structurally variant polypeptide molecules produced by the method as described herein, and b) determining at least one property of the plurality of folded structurally variant polypeptide molecules.

### BRIEF DESCRIPTION OF THE FIGURES AND TABLES

FIG. 1. Design and evaluation of a streamlined process for rapid and inexpensive production of large panels of chemokine analogs.

(Left panel)Prior art work exploring chemokine structure-activity relationships has been based on chemical synthesis of individual molecules. In an embodiment of the present invention (right panel) a process for the parallel production of polypeptide variants is provided. Solid boxes [symbol] : steps performed in series, Dotted/Dashed boxes [symbol] : parallel steps, Dashed boxes [symbol] : column chromatography steps, Dotted boxes [symbol] : parallel procedures used to replace column chromatography steps.

FIGS. 2, 2A, and 2B. Analysis of parallel synthesized chemokine analogs produced using core Fragment batch 1 by RP-HPLC.

Following the final step of synthesis, samples from each reaction were subjected to analytic RP-HPLC. For each panel, x-axis is time in minutes, y-axis is UV absorbance (AU 214 nm).

FIGS. 3 and 3A. Analysis of parallel synthesized chemokine analogs produced using core Fragment batch 2 by RP-HPLC.

Following the final step of synthesis, samples from each reaction were subjected to analytic RP-HPLC. For each panel, x-axis is time in minutes, y-axis is UV absorbance (AU 214 nm).

FIG. 4. In syntheses yielding two major products the peak with the shorter RP-HPLC retention time has a mass corresponding to the Met⁶⁷(O) congener of the target product.

Two completed parallel synthesis reactions yielding two major product peaks were subjected to analytical RP-HPLC and with each peak collected and analyzed by MALDI MS. Observed masses for the peaks with the longer retention time were consistent with those of the target product (expected masses of 7987 Da and 7891 Da for 2P14-RANTES and 8P2-RANTES, respectively), and those of peaks with the shorter retention time were consistent with those of the Met⁶⁷(O) congeners of the target products (mass differences between the shorter and longer retention time peaks of +17 Da and +13 Da for 2P14-RANTES and 8P2-RANTES, respectively).

FIG. 5. Comparison of RP-HPLC retention times of parallel synthesis products with reference standard chemokine analogs.

The RP-HPLC profiles of representative parallel synthesis products (inwell mixture) were compared with those of reference standard chemokine analogs (ref. std from Gaertner 2008) using the same analytical column and under identical conditions. Peak retention times for major peaks are noted for each sample. A. Representative samples produced using Core Fragment batch 1, B. Representative samples produced using Core Fragment batch 2.

FIG. 6. Comparison of the anti-HIV potencies of parallel-synthesized chemokine analogs with those previously obtained for the corresponding reference standard samples.

A. Examples of data used for stratification, R5-tropic envelope-dependent cell fusion assays were performed at four different nominal concentrations, with samples ranked from (−) to (++++) according to the number of concentrations at which complete inhibition of cell fusion was achieved. Symbols indicate mean cell fusion activity±range (n=3). Black squares: M9-RANTES, black triangles: M19-RANTES, black circles: 8P5-RANTES, white squares: 8P6-RANTES, white triangles: M21-RANTES, white circles: 5P12-RANTES reference standard. B. The potency of each compound produced and stratified in this study compared to the potency (pIC50) of the corresponding molecule produced and tested in the reference study (Gaertner 2008).

FIG. 7. Assessment of Ca2+ signaling activity of parallel-synthesized chemokine analogs.

The signaling activity of each analog synthesized in this study was determined using a 384-well format Ca2+ flux assay on HEK-CCR5 cells loaded with Fluo4-AM. Analogs were tested at a nominal Eₘₐₓ concentration of 300 nM, together with reference standard samples (300 nM) of 5P12-RANTES (nonsignaling control) and PSC-RANTES (maximum signaling control). Percentage signaling was calculated as follows: 100×(Sample signal −5P12-RANTES signal)÷(PSC-RANTES signal −5P12-RANTES signal). Bars indicate mean±SEM (n=4).

FIG. 8. Comparison of the calcium signaling activity of parallel-synthesized chemokine analogs with those previously obtained for the corresponding reference standard samples.

The calcium signaling assay of each parallel-synthesized chemokine analog was determined at an Eₘₐₓ concentration (300 nM) in a 384-well plate based assay (see FIG. 7). According the results obtained, analogs were stratified into three groups (low, medium and high signaling). This figure shows the distribution of signaling efficacies determined using molecules produced and tested in the reference study (Gaertner 2008) for the analogs in each group.

FIG. 9. Assessment of CCR5 internalization activity of parallel-synthesized chemokine analogs.

The CCR5 internalization activity of each analog synthesized according to the method of the present invention was determined using a 96-well format bystander BRET assay on CHO-CCR5-RLuc8/YFP-CAAX reporter cells. Analogs were tested at a nominal Eₘₐₓ concentration of 300 nM, together with reference standard samples (300 nM) of 5P12-RANTES (non-internalizing control) and PSC-RANTES (maximum internalizing control). Percentage signaling was calculated as follows: 100×(Sample signal −5P12-RANTES signal)÷(PSC-RANTES signal−5P12-RANTES signal). Bars indicate mean±SEM (n=6).

FIG. 10. Comparison of the CCR5 downmodulation activity of parallel-synthesized chemokine analogs with those previously obtained for the corresponding reference standard samples.

The CCR5 downmodulation activity of each parallel-synthesized chemokine analog was determined at an Eₘₐₓ concentration (300 nM) in a BRET-bystander assay (see FIG. 9). According the results obtained, analogs were stratified into three groups (low, medium and high downmodulation). This figure shows the distribution of downmodulation efficacies determined using molecules produced and tested in the reference study (Gaertner 2008) for the analogs in each group.

FIG. 11-11B. Analysis of target CCL25 analogs by HPLC.

Following synthesis, the CCL25 analogs were analyzed by HPLC.

FIG. 12. Assessment of the ability of CCL25 analogs to recruit arrestin-3 to CCR9 by bioluminescence resonance energy transfer.

BRET signals (mean and std dev, n=4) obtained for CCL25 analogs (300 nM) on CCR9-expressing reporter cells. The dotted horizontal line represents background signaling level.

Table 1. MALDI MS analysis of parallel synthesized N-terminal peptide fragments.

Following synthesis, of each N-terminal-SEA fragment was analyzed by MALDI MS. Sequences and expected masses of each fragment, together with observed masses, mass difference and interpretation are shown.

Table 2A. MALDI MS analysis of parallel chemokine analogs produced using Core Fragment batch 1.

Following the final step of synthesis, samples from each reaction were analyzed by MALDI MS. Expected and observed masses for each analog are shown.

Table 2B. MALDI MS analysis of parallel chemokine analogs produced using core Fragment batch 2.

Following the final step of synthesis, samples from each reaction were analyzed by MALDI MS. In the majority of cases, two major masses were detected. These masses are presented (Mobs1 and Mobs2) together with the expected mass of each analog.

Table 3. Estimating target protein concentration in samples of parallel-synthesized chemokine analogs.

Following the final step of synthesis, samples from selected reactions were dissolved in 250 μL water and subjected to analytical RP-HPLC and the percentage areas of peaks interpreted as authentic target protein and its Met⁶⁷(O) congener were calculated using Empower software (Waters). Total protein concentrations (μM) and content (nmol) were estimated by measuring the absorbance (280 nm) of each solution and using the theoretical extinction coefficient of each analog (web.expasy.org/protparam). Estimated % yields of target protein were calculated as 100×combined % target peak area×estimated total protein contents (nmol)÷100 nmol (amount of Core Fragment in initial reaction). Estimated concentrations in working solutions (250 μL) were calculated by multiplying the estimated total peptide concentration (determined by absorbance at 280 nm) by the estimated % yield of target protein.

Table 4. Variant Region N-terminal peptides of CCL25 analogs.

N-terminal sequences of synthesized CCL25 analogs. The sequences shown are N-terminal to Cys8 of native CCL25. Certain analogs feature N-terminal extensions. Z=pyroglutamate.

Table 5. MALDI MS analysis of target CCL25 analogs.

Following synthesis, the CCL25 analogs were analyzed by MALDI mass spectrometry.

### DETAILED DESCRIPTION

Described in the present application is a method for producing a large number of variant polypeptide molecules in parallel and in a form suitable for determining their effects and/or properties by subsequent analysis. The economical and parallel nature of the present invention allows for large numbers of polypeptide molecules to be screened quickly and efficiently for biological or medical research.

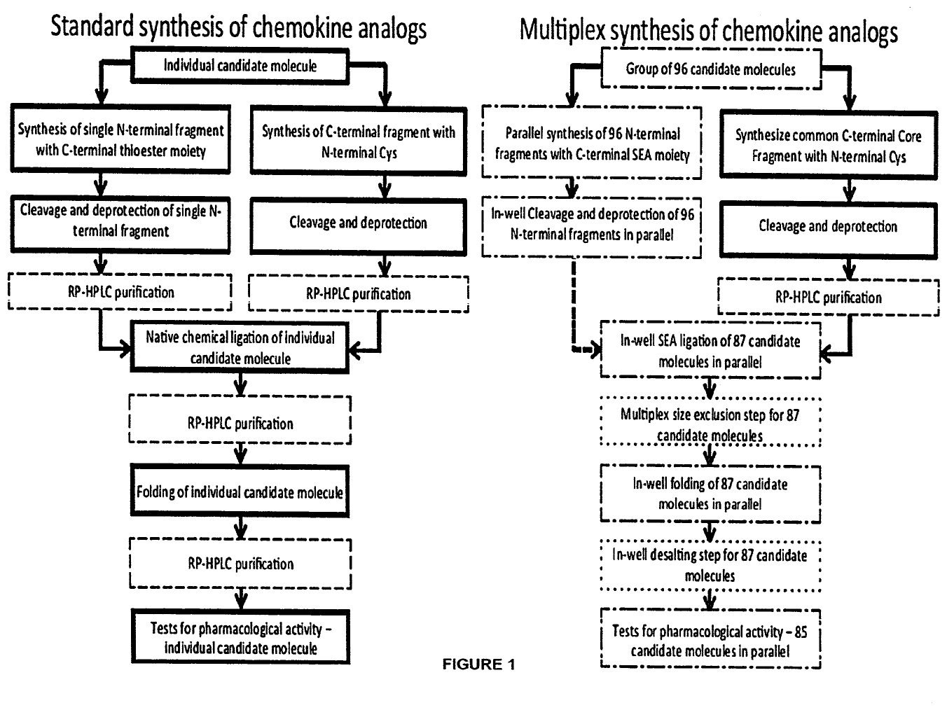

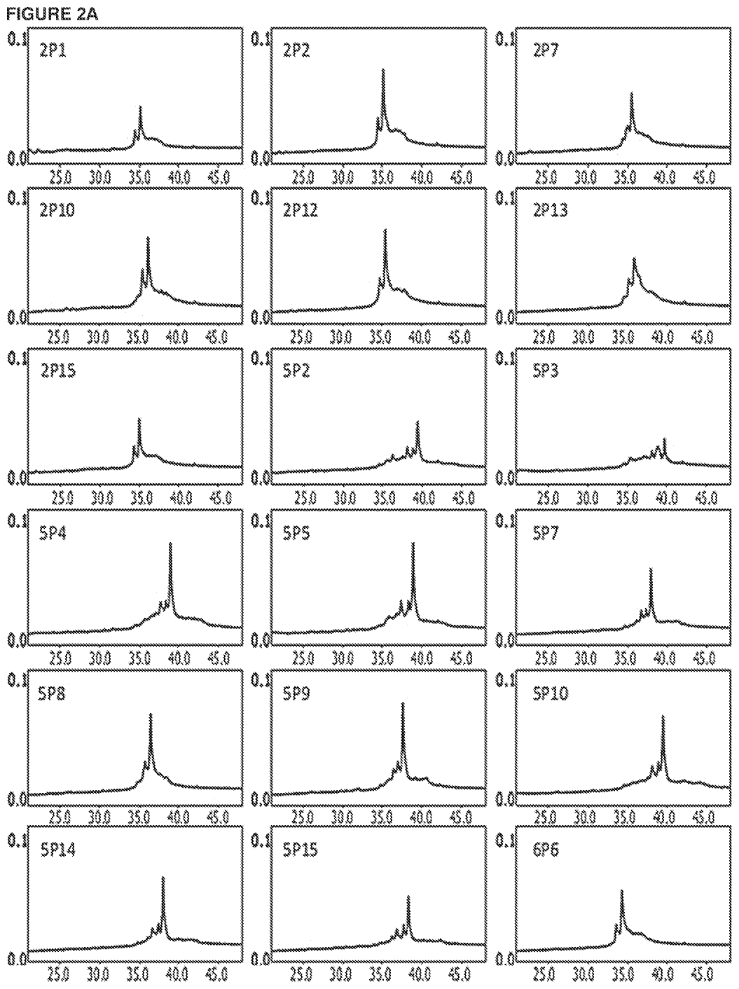

In an embodiment of the present invention, described herein in Examples 1-4 and provided as non-limiting examples, 96 candidate variants of the chemokine RANTES/CCL5 were selected for production and subsequent analysis by the present method. These 96 variants represent analogs of the RANTES/CCL5 protein (Gaertner 2008). Two large batches of an invariant Core Fragment of RANTES/CCL5 were produced by solid phase peptide synthesis. As well, an array of variant peptides corresponding to the variant region of RANTES/CCL5 was produced in parallel by solid phase peptide synthesis. The Core Fragment was ligated in parallel to each of the variant peptides to produce an array of structurally variant RANTES/CCL5 analogs. The ligated RANTES/CCL5 analogs were then separated from the ligation reaction mixtures by size exclusion but without the use of column chromatography, allowing for the procedure to be completed quickly and in parallel. The RANTES/CCL5 analogs were then folded, desalted, and lyophilized in parallel. A flow chart depicting the steps of this embodiment of the present invention compared to previous methods known in the art is provided in a non-limiting example in FIG. 1.

In another embodiment of the present invention, described herein in Examples 5-7 and provided as non-limiting examples of the present invention, a selection of the RANTES/CCL5 analogs produced by the present method were selected for analysis of their biological effects on cells and compared to data previously described for these same analogs when produced by a more laborious and costly method using HPLC purification (Gaertner 2008). The method was successful in generating 85 RANTES/CCL5 analogs in parallel for analysis. These RANTES/CCL5 analogs were then applied to cells and assayed for their biological activity by cell fusion assay (FIG. 6), CCR5 agonist assay (FIG. 7 and FIG. 8), and cell surface downmodulation assay (FIG. 9 and FIG. 10). As shown herein, analysis of the cellular effects of the analogs produced by the present method obtained a good correlation with the effects of the same analogs produced by a previously described method (Gaertner 2008). These results were surprising; although the analogs produced by the present method were of lower purity than those produced by known methods using column chromatography (Canne US 7,094,871) or tag-based resin-capture (Loibl 2016) steps, they were still able to accurately recapitulate their biological effects in 3 cellular assays.

In another embodiment of the present invention, described herein in Example 8, and provided as a non-limiting example of the present invention, 50 analogs of CCL25 were produced and subsequently analyzed for biological activity by the present method. The method was successful in generating 50 CCL25 analogs that were screened for their biological activity in a cellular assay that measured the ability of the analogs to recruit arrestin-3 to CCR9 on CCR9-expressing reporter cells. Some of the analogs exhibited higher activity than the parent compound (FIG. 12).

The examples described herein indicate that polypeptide variants produced by the present method can be reliably screened for their effects and/or properties. Furthermore, the present method can produce these screenable polypeptide variants in large numbers and in parallel without the need for the costly and limiting purification procedures required in known methods (Canne U.S. Pat. No. 7,094,871; Low WO2004105685; Loibl 2016).

Polypeptides

In the present invention, a plurality of structurally variant polypeptides is provided, as well as a common invariant polypeptide, for use in the production of a plurality of structurally variant polypeptide molecules.

The term “structurally variant”, as used herein, refers to at least one variation in the structure of a polypeptide relative to other corresponding or analogous polypeptides. The structural variation can be, for example, at least one change in the amino acid sequence relative to the other variants, such as a deletion, insertion, replacement, or modification. The structural variation can also be, for example, the incorporation of at least one amino acid analog, amino acid derivative, or non-amino acid moiety. The structural variations present among structurally variant polypeptides are likely to alter the properties and/or effects of the final polypeptide molecule in a manner that can be detected in an assay as described herein.

The term “structurally invariant”, as used herein, refers to a polypeptide that contains no variations, relative to other corresponding or analogous polypeptides, that significantly alter the properties and/or effects of the final polypeptide molecule. Invariant polypeptides may be identical or may contain, for example, conservative amino acid substitutions that do not affect the conformation or function of the polypeptide or, in another example, may contain modifications at sites that are known not to be involved in target binding. Any variations among common, structurally invariant polypeptides should not alter the properties and/or effects of the final polypeptide molecule, such that any differences in the properties and/or effects among the plurality of structurally variant polypeptide molecules are attributable to the structurally variant regions.

The term “parallel”, as defined herein, refers to 2 or more polypeptides being any one or more of produced, synthesized, ligated, folded, desalted, reacted, separated, lyophilized, or otherwise manipulated at once. Polypeptides can be produced, synthesized, ligated, folded, desalted, reacted, separated, lyophilized, or otherwise manipulated, for example, in parallel in groups of 2, 4, 6, 8, 12, 24, 48, 96, 192, 288, 384, or more.

The synthesis of polypeptides can be performed individually using a peptide synthesizer with a single reaction vessel, such as an ABI 433 Peptide Synthesizer (Applied Biosystems). A non-limiting example of this is the synthesis of the Core Fragment Batch 1, disclosed herein in Example 1.

The synthesis of polypeptides can also be performed in parallel in groups of, for example, 2, 4, 6, 8, 12, 24, 48, 96, 192, 288, 384, or more. Use of a Prelude® peptide synthesizer (Protein Technologies Inc.), for example, allows for the synthesis of 1-6 separate polypeptides in parallel. The use of an ABI 433 Peptide Synthesizer (Applied Biosystems), for example, allows for the synthesis of polypeptides in parallel in groups of 48, 72, 96, 192, 288, and 384. A non-limiting example of this is the parallel synthesis of 96 Variant Region peptides, disclosed herein in Example 2. Example 2 discloses, by way of a non-limiting example, how the present invention can provide a plurality of structurally variant polypeptide regions in a column-free manner for use in a subsequent ligation reaction.

Reaction vessels suitable for the parallel synthesis of polypeptides, as described above, include, but are not limited to: blocks of 12, 24, 48, or 72 columns or tubes, 96-well plates, and 384-well plates.

Polypeptides for use in the invention can be synthesized, whole or in part, by linking amino acids using chemical methods known in the art. For example, peptide synthesis can be performed using various solid-phase techniques (see e.g., Roberge 1995; Merrifield 1997; 011ivier 2010; Raibaut 2015). Solid-phase peptide synthesis can employ either Foc or Bmoc chemistries as known in the art (Jaradat 2018). Furthermore, automated synthesis may be achieved using, for example but not limited to, the ABI 433 Peptide Synthesizer (Applied Biosystems), the Prelude® synthesizer (Protein Technologies Inc.), or the MultiPep RSi 384-well peptide synthesizer (Intavis) in accordance with the instructions provided by the manufacturer. Polypeptides for use in the present invention can be synthesized as disclosed herein in Examples 1 and 2.

It is also contemplated by the inventors that peptides for use in the invention can be synthesized in parallel by other methods known in the art, including laser-based techniques (Loeffler 2016) or flow-based techniques (Mijalis 2017).

Polypeptides are polymers of amino acids linked covalently by peptide bonds. Short polypeptides of <10, <15, <20, or <50 amino acids in length are often referred to in the art as “peptides”. Longer polypeptides of >10, >15, >20, or >50 amino acids in length are often referred to in the art as “polypeptides”. As used herein, the term “polypeptide” is used to describe any polymer comprising 2 or more amino acids.

Polypeptides for use in the present invention can be synthesized, for example, to lengths of 2, 4, 5, 6, 8, 10, 12, 15, 20, 25, 30, 35, 40, 45, or 50 amino acids.

In some embodiments of the present invention, the plurality of structurally variant polypeptides correspond to a structurally variant region of a polypeptide molecule.

In some embodiments of the present invention, the common invariant polypeptide corresponds to a structurally invariant region of a polypeptide molecule.

In some embodiments of the present invention, the plurality of structurally variant polypeptides and the common invariant polypeptide correspond to a structurally variant region and the structurally invariant region, respectively, of the same polypeptide molecule.

In some embodiments of the present invention, the plurality of structurally variant polypeptides and/or the common invariant peptide are synthesized by solid-phase peptide synthesis. Solid-phase peptide synthesis can be performed according to known methods (see e.g. Roberge 1995; Merrifield 1997; Raibaut 2015).

In some embodiments of the present invention, the plurality of structurally variant polypeptides are synthesized by solid-phase peptide synthesis in parallel. Solid-phase peptide synthesis can be done in parallel using, for example but not limited to, a MultiPep RSi 384-well peptide synthesizer.

Polypeptides are polymers that comprise amino acids linked by peptide bonds. As used herein, the term “amino acid” is used to describe any amino acid, natural or otherwise, that can be incorporated into a polypeptide. Amino acids are small molecules comprising an amine (—NH₂) group, a carboxyl (—COOH), and a variable side chain (R-group) specific to each amino acid. Amino acids are covalently linked by peptide bonds between the amine group of one amino acid to the carboxyl group of another amino acid to form polypeptides. Amino acids within a polypeptide are often referred to in the art as “residues”.

In some embodiments, the structurally variant polypeptides and/or the common invariant polypeptide comprise amino acid analogs. As used herein, the term “amino acid analogs” is used to describe artificial, synthetic, or unnatural amino acids beyond the canonical 20 genetically-encoded amino acids (Zou 2018).

Amino acid analogs for use in the invention can be synthesized by known methods (see e.g. Zou 2018; Boto 2007; He 2014) or can be purchased from a known supplier (Millipore Sigma).

Examples of amino acid analogs that can be incorporated into polypeptides in some embodiments include, but are not limited to, β-amino acids, homo-amino acids, synthetic proline and pyruvic acid derivatives, 3-substituted alanine derivatives, glycine derivatives, ring-substituted phenylalanine and tyrosine derivatives, linear core amino acids, N-methyl amino acids, and amino acids with synthetic R-groups. In some embodiments, the structurally variant polypeptides and/or the common invariant polypeptide comprise amino acid derivatives. As used herein, the term “amino acid derivatives” is used to describe amino acids that have been derived from the modification of one of the canonical 20 genetically-encoded amino acids. Amino acid derivatives can be synthetic, e.g. made in vitro by chemical reaction, or they can be naturally occurring in organisms, e.g. in vivo metabolites. An example of an amino acid derivative is pyroglutamate/pyroglutamic acid, a cyclized derivative of glutamine in which the free amino group of glutamic acid cyclizes to form a lactam.

In some embodiments, a molecular hook that enables the attachment of labeling structures including, but not limited to, fluorochromes, chelators, and biotin, can be incorporated into the structurally variant polypeptide molecules. By way of non-limiting example, during synthesis of the invariant core fragment it is possible to introduce a moiety that enables the generation of site-specific modified variants of the core fragment derivatized with a wide range of useful structures, in particular those that could be used for detection (e.g. fluorochromes) and those that could be used for purification (e.g. biotin). Such a moiety can be incorporated, for example, via the epsilon amine of a chosen lysine residue in the polypeptide, Moieties that can be incorporated include, but are not limited to, (i) unnatural amino acids containing side chains compatible with ‘click chemistry’ (Kolb, 2001), and (ii) serine resides that can be oxidized to generate an aldehyde functionality that is compatible with oxime chemistry.

In some embodiments, the polypeptides incorporate moieties that are not amino acids. Polypeptides containing moieties that are not amino acids are suitable for use in the methods of the present invention as long as they can be synthesized and ligated using synthesis and ligation techniques as disclosed herein.

Proteins and Protein Analogs

The present invention provides a method for producing a plurality of structurally variant polypeptide molecules. Proteins are a class of biological molecule comprised primarily of one or more polypeptides. As used herein, the term “protein” is used to describe a molecule comprising one or more polypeptides.

If a protein comprises more than one polypeptide, said polypeptides may be covalently or non-covalently linked. As well, a polypeptide in a protein may be covalently linked back unto itself through a covalent bond between two R-groups, e.g. a disulfide bridge. The polypeptides in a protein may be modified to include a lipid molecule (lipopeptides and lipoproteins) or a carbohydrate molecule (glycopeptides and glycoproteins). As well, proteins may be linked with a non-organic component (e.g. an iron atom in the heme group of the hemoglobin protein).

In some embodiments of the invention, the plurality of structurally variant polypeptides correspond to a structurally variant region of a protein.

In some embodiments, the common invariant polypeptide corresponds to a structurally invariant region of a protein.

In some embodiments, the plurality of structurally variant polypeptides and the common invariant polypeptide correspond to a structurally variant region and a structurally invariant region, respectively, of the same protein.

In some cases, a protein may exist in numerous structurally variant forms. These structurally variant forms of a protein are often referred to in the art as “analogs”. These protein analogs share one or more common, structurally invariant regions, but differ in one or more structurally variant regions. The protein analogs may share certain effects or properties, based on their shared invariant region(s), and may also exhibit differential effects or properties due to differences imparted by the variant regions.

In embodiments where the plurality of structurally variant polypeptides corresponds to a region of a known polypeptide or protein, they can be ligated to a common invariant polypeptide that corresponds to a region of the same polypeptide or protein to produce a plurality of analogs of said polypeptide or protein.

In embodiments where the plurality of structurally variant polypeptides corresponds to a region of a known polypeptide or protein, they can be ligated to a common invariant polypeptide that corresponds to a region of a different polypeptide or protein to produce a plurality of analogs of a chimeric polypeptide or protein.

In some embodiments, the plurality of structurally variant polypeptides and/or the common invariant polypeptide can be artificial polypeptides that do not correspond to regions any known proteins. In some embodiments, the artificial structurally variant polypeptides can be ligated to an artificial common invariant polypeptide to produce a plurality of analogs of an artificial polypeptide or protein. In some embodiments, the artificial structurally variant polypeptides can be ligated to a common invariant polypeptide that corresponds to a known region of a polypeptide or protein to produce a plurality of analogs of a chimeric polypeptide or protein. In some embodiments, an artificial common invariant polypeptide can be ligated to artificial structurally variant polypeptides that correspond to a known region of a polypeptide or protein to produce a plurality of analogs of a chimeric polypeptide or protein.

In some embodiments of the present invention, the structurally variant polypeptide molecules produced by the method are proteins. Examples of proteins that could be produced by the present invention include, but are not limited to, transcription factors, transcription enhancers, transcription repressors, DNA/RNA-binding proteins, complement fragments, cytokines, chemokines, cell surface receptor domains, intracellular receptors, enzymes, antibody fragments, hormones, toxins, individual protein domains, and artificial proteins. Artificial proteins can include polypeptides wherein the structurally variant regions are designed to be mimics of small molecules and other non-polypeptide ligands (e.g. nucleotides, polysaccharides, or lipids), and the structurally variant regions are ligated to an artificial common invariant region or a common invariant region derived from a known protein. Cytokines are a class of proteins important to the immune system. Cytokines allow immune cells to signal to one another to coordinate immune responses.

In some embodiments of the invention, the structurally variant polypeptide molecules produced by the method are cytokines. Examples of cytokines that could be produced by the present invention include, but are not limited to, IL-1, IL-2, IL-3, IL-4, IL-5, IL-6, IL-10, interferon-alpha, interferon-gamma, tumour necrosis factor alpha, and tumour growth factor-beta.

Chemokines are a specific class of cytokines that recruit cells to specific locations by inducing chemotaxis by signaling through chemokine receptors. Chemokines are classified into four groups (C chemokines, CC chemokines, CXC chemokines, and CXXXC chemokines). An example of a chemokine is RANTES (regulated on activation, normal T cell expressed and secreted), also known as CCL5 (C-C motif ligand 5). RANTES/CCL5 binds to the receptor CCR5 to induce chemotaxis and promote immune responses. Analogs of RANTES/CCL5 that are useful for treatment of HIV are known in the art (Gaertner 2008). RANTES/CCL5 is disclosed herein as a non-limiting example of a chemokine protein suitable for use in the present invention in Examples 1-7. Another example of a chemokine is CCL25, which binds to the receptor CCR9. CCL25 is expressed, for example, by intestinal epithelial cells and promotes the recruitment of CCR9-expressing lymphocytes. CCL25 is disclosed herein as a non-limiting example of a chemokine protein suitable for use in the present invention in Example 8.

In some embodiments of the invention, the structurally variant polypeptide molecules produced by the method are chemokines. Chemokines that could be produced by the present invention include, without limitation, C chemokines, CC chemokines, CXC chemokines, and CXXXC chemokines. Chemokines that could be produced by the present invention include, without limitation, homeostatic and inflammatory chemokines.

Ligation of Polypeptides

The present invention provides a method for producing a plurality of structurally variant polypeptide molecules in which a plurality of structurally variant polypeptides is ligated to a common invariant polypeptide.

Ligation of polypeptides can be performed by a number of techniques known in the art including imine capture, pseudoproline ligation, Staudinger ligation, thioester capture ligation, and hydrazine formation ligation (Tam 2001).

Ligation of polypeptides can be performed by native chemical ligation as known in the art (Dawson 1994; Raibaut 2015; Engelhard 2016). Native chemical ligation allows the covalent assembly of two or more unprotected peptide segments to produce a larger polypeptide. Native chemical ligation reactions can occur as soluble ligations, in which the polypeptides to be conjugated are in solution, or as solid-phase ligations, in which the N-terminal polypeptide fragment is covalently attached to a solid-phase resin via a detachable linker (Canne U.S. Pat. No. 7,094,871; Low WO2004105685).

Ligation of polypeptides can be performed by SEA native peptide ligation as known in the art (011ivier 2010). In this method, ligation occurs between the C-terminal bis(2-sulfanylethyl)amido (SEA) group of one peptide and the N-terminal cysteine of another peptide. Similarly to native chemical ligation, SEA native peptide ligation can occur as a soluble or as a solid-phase ligation reaction. The use of SEA native peptide ligation in the present invention is disclosed in a non-limiting example of an embodiment of the present invention in Example 3.

In some embodiments of the present invention, the ligation reaction between a plurality of structurally variant polypeptides and a common invariant polypeptide occurs via native chemical ligation.

In some embodiments of the present invention, the ligation reaction between a plurality of structurally variant polypeptides and a common invariant polypeptide occurs via SEA native peptide ligation. SEA native peptide ligation can be perfomed according to techniques known in the art (011ivier 2010)

In some embodiments of the present invention, the ligation reaction between a plurality of structurally variant polypeptides and a common invariant polypeptide occurs via soluble ligation.

In some embodiments of the present invention, the ligation reaction between a plurality of structurally variant polypeptides and a common invariant polypeptide occurs via a soluble SEA native peptide ligation.

In some embodiments of the present invention, the ligation reaction between a plurality of structurally variant polypeptides and a common invariant polypeptide occurs via a soluble SEA native peptide ligation in parallel.

The ligation of polypeptides can be performed in parallel in groups of, for example, 2, 4, 6, 8, 12, 24, 48, 96, 192, 288, 384, or more. Ligation can be performed in parallel by applying ligation reaction mixtures in parallel in suitable vessels. Such vessels include, but are not limited to, 0.2 mL tubes, 0.5 mL tubes, 1.5 mL tubes, 2 mL tubes, 15 mL tubes, 48-well plates, 96-well plates, and 384-well plates.

In some embodiments of the present invention, the structurally variant polypeptides correspond to the N-terminal region of a polypeptide molecule.

In some embodiments of the present invention, the structurally variant polypeptides correspond to the C-terminal region of a polypeptide molecule.

In some embodiments of the present invention, the structurally variant polypeptides correspond to an internal region of a polypeptide molecule.

In some embodiments of the present invention, the common invariant polypeptide corresponds to the N-terminal region of a polypeptide molecule.

In some embodiments of the present invention, the common invariant polypeptide corresponds to the C-terminal region of a polypeptide molecule.

In some embodiments of the present invention, the common invariant polypeptide corresponds to an internal region of a polypeptide molecule.

In some embodiments of the present invention, the polypeptide molecule is a protein.

Size Exclusion, Reaction Separation, and Column Chromatography

The present invention provides a method for producing a plurality of structurally variant polypeptide molecules by ligation of two polypeptide fragments in which said polypeptide molecules are separated from the ligation reaction solution after ligation.

Numerous techniques are known in the art for separating a molecule from a reaction mixture and/or from incomplete reaction products. These techniques can be important, as chemicals in the reaction mixture, or incomplete reaction products, can interfere with downstream uses of a desired reaction product. By way of example, the reaction mixture of a completed SEA native peptide ligation contains thiol scavengers, reducing agents, and unreacted N-terminal peptides that would interfere with the downstream folding of a polypeptide ligation product. It is therefore important that these unwanted reaction constituents be removed by purifying the reaction product.

The most robust and effective technique for separating polypeptide molecules from a reaction mixture is high performance liquid chromatography (HPLC). HPLC is also commonly known in the art as column chromatography. Column chromatography involves pumping a solution containing the molecule(s) to be separated into a column containing a solid-phase. The properties of the solid phase determine the specific column chromatography technique and the mechanism by which the molecule(s) are separated. Examples of column chromatography include size-exclusion chromatography, normal-phase chromatography, reversed-phase chromatography, and affinity chromatography. Of particular relevance is reversed-phase chromatography, in which a hydrophobic solid-phase is used to adsorb polypeptides while other solutes and solvents pass through the column. All of these methods of column chromatography are known in the art and can be used to achieve a high purity of a desired molecule (see Snyder 2000). One major disadvantage of column chromatography is the cost in both resources and time. Column chromatography can be a slow process, and only one sample can be run through a column at a time. Machines that perform column chromatography are relatively large and costly, and it is logistically prohibitive to run large numbers of them in parallel. Therefore, the requirement for column chromatography in any method for producing or analyzing polypeptides is a rate-limiting step that prevents large-scale parallel operation.

Other techniques are known in the art for separating a polypeptide molecule from a reaction mixture. These techniques are commonly known as “column-free” to distinguish them from column-based techniques such as HPLC. Column-free techniques of particular suitability to the present invention are column-free reverse phase separation, membrane filtration, precipitation/centrifugation, and dialysis. Although column-free purification techniques do not achieve the same high purity of final product as HPLC, they do have an advantage in time, cost, and scalability, and are therefore well-suited for use in parallel.

Membrane filtration is a column-free size-exclusion technique whereby the solution containing the desired molecule is applied to a membrane or filter with a defined pore size. The pore size of a membrane or filter is often defined in the art by cut-off size, using the unit kilodalton (kDa). The cut-off size in kDa indicates that all liquids, solutes, and molecules with a kDa smaller than the cut-off size will pass through the membrane or filter. Conversely, all molecules with a kDa larger than the cut-off size will be retained by the membrane or filter. In this manner, the molecule of interest is separated from a solution or reaction mixture. Examples of cut-off sizes for membranes or filters used in purifying polypeptides include, but are not limited to 3.5 kDa, 10 kDa, 30 kDa, and 50 kDa.

Examples of membrane filtration vessels that are suitable for separation of polypeptide molecules include, but are not limited to, Microcon® Tubes (Millipore Sigma), Amicon Ultra Tubes (Millipore Sigma), and 96-well MultiScreen™ filter plates (Millipore Sigma). The use of 10 kDa cut-off Microcon® tubes for parallel polypeptide purification in the present invention is disclosed by way of a non-limiting example herein in Example 3.

The separation of polypeptides from reaction mixtures by membrane filtration can be performed in parallel in groups of, for example, 2, 4, 6, 8, 12, 24, 48, 96, 192, 288, 384, or more. Separation can be performed in parallel by applying reaction mixtures containing the polypeptides of interest in parallel in suitable vessels. Such vessels include, but are not limited to, Microcon® Tubes (Millipore Sigma), Amicon Ultra Tubes (Millipore Sigma), and 96-well MultiScreen™ filter plates (Millipore Sigma).

In membrane filtration techniques, solutions containing the desired molecule can be applied without pressure and allowed to run through the membrane or filter by the force of gravity alone. Alternatively, solutions containing the desired molecule can be applied to the membrane or filter with pressure to force the solution through. Pressure can be applied through the use of a centrifuge or a pump.

When performing membrane filtration, the solution will flow through the membrane or filter and can be discarded, and the desired molecule will be retained by the filter. The desired molecule can then be subjected to washing steps by repeatedly applying a washing solution to the membrane or filter and allowing or forcing the washing solution through the membrane or filter. Suitable washing solutions include, but are not limited to, water and guanidine hydrochloride.

When performing membrane filtration, the desired molecule will be retained by the membrane or filter and can be recovered by removing the liquid containing the desired molecule directly. If there is not sufficient liquid on the membrane or filter, the desired molecule can be recovered by applying a suitable solvent to the membrane or filter and then removing the solvent to recover the desired molecule in solution. Suitable solvents for resuspending a desired polypeptide from a membrane or filter include, but are not limited to, water, guanidine hydrochloride, and folding buffer.

Another suitable column-free technique for separating a polypeptide from a reaction mixture is precipitation/centrifugation, in which one or more of the undesirable contaminants precipitates while the desired molecule remains in solution and can be isolated by centrifugation and removal of the soluble phase. Such a technique can be performed, for example, by a) adding two volumes of 6M guanidine hydrochloride solution supplemented with TCEP (0.2M) to each 100 uL ligation mixture, b) acidifying the reaction with 50 uL of 33% acetic acid, c) adding 4 mL of 2M guanidine hydrochloride solution to precipitate MPAA scavengers, d) centrifuge the reaction at 2000 g for 5 min, and e) removing the supernatant for subsequent reverse-phase extraction. The method for purification by precipitation/centrifugation provided herein is for 100 uL reaction volumes. It will be achievable for a person skilled in the art to modify this method for use with other reaction volumes.

The separation of polypeptides from reaction mixtures by precipitation/centrifugation can be performed in parallel in groups of, for example, 2, 4, 6, 8, 12, 24, 48, 96, 192, 288, 384, or more. Separation can be performed in parallel by applying precipitation conditions as described in [00110] to reaction mixtures containing the polypeptides of interest in parallel in suitable vessels. Such vessels include, but not limited to, 0.2 mL tubes, 0.5 mL tubes, 1.5 mL tubes, 2 mL tubes, 15 mL tubes, 48-well plates, 96-well plates, and 384-well plates.

Another suitable column-free technique for the separation of a polypeptide from a reaction mixture is dialysis. Dialysis is a technique whereby the solution containing the molecule of interest (solution 1) is placed is a vessel with one or more porous surfaces. The pores of the vessel are of a size specified by kDa cut-off. The molecule of interest will be retained in the vessel because it is larger than the pores as defined by kDa cut-off. The vessel is then placed in a volume of a different, desired solution (solution 2). The undesired solvents and solutes of solution 1 will pass out of the vessel through the pores by the process of osmosis and, likewise, the desired solution 2 will flow through the pores into the vessel. Thereby, the molecule of interest will be separated from solution 1 (e.g. the ligation reaction) by osmosis.

Suitable vessels for separation by dialysis include, but are not limited to, dialysis tubing, Slide-A-Lyzer™ dialysis casettes (ThermoFisher), Pur-A-Lyzer™ dialysis kits (Millipore Sigma), and Pierce™ 96-well Microdialysis plates (ThermoFisher).

Suitable kDa cut-offs for dialysis of polypeptide molecules include, but are not limited to, 3.5 kDa, 6 kDA, 8 kDa, 10 kDa, 12 kDa, 14 kDa, and 20 kDa.

The separation of polypeptides from reaction mixtures by dialysis can be performed in parallel in groups of, for example, 2, 4, 6, 8, 12, 24, 48, 96, 192, 288, 384, or more. Separation can be performed in parallel by applying reaction mixtures containing the polypeptides of interest in parallel in suitable dialysis vessels. Such vessels include, but are not limited to, dialysis tubing, Slide-A-Lyzer™ dialysis casettes (ThermoFisher), Pur-A-Lyzer™ dialysis kits (Millipore Sigma), and Pierce™ 96-well Microdialysis plates (ThermoFisher).

In some embodiments of the present invention, the plurality of structurally variant polypeptide molecules is separated from the ligation reaction by membrane filtration.

In some embodiments of the present invention, the plurality of structurally variant polypeptide molecules is separated from the ligation reaction by membrane filtration in parallel.

In some embodiments of the present invention, the plurality of structurally variant polypeptide molecules is separated from the ligation reaction by precipitation/centrifugation.

In some embodiments of the present invention, the plurality of structurally variant polypeptide molecules is separated from the ligation reaction by precipitation/centrifugation in parallel.

In some embodiments of the present invention, the plurality of structurally variant polypeptide molecules is separated from the ligation reaction by dialysis.

In some embodiments of the present invention, the plurality of structurally variant polypeptide molecules is separated from the ligation reaction by dialysis in parallel.

Protein Folding

The present invention provides a method for producing a plurality of structurally variant polypeptide molecules in which said plurality of structurally variant polypeptide molecules is folded.

Proteins are characterized by having four layers of structure. The primary structure of a protein is encoded by its linear amino acid sequence. The secondary structure of a protein is formed by localized by hydrogen bonding between amino acids to form sub-structures called α-helices and β-sheets, as well as non-structured units known as random coils. The tertiary structure of a protein is formed by the 3-dimensional folding of the α-helices, β-sheets, and random coils into a globular structure. Folding of the protein into a tertiary structure is caused by hydrogen bonds, salt bridges, hydrophobic interactions, and covalent disulfide bridges. Finally, the quaternary structure of a protein is formed by the association of multiple polypeptides and other non-organic groups.

As used herein, the term “folded” is used to describe a protein, or a region of a protein corresponding to one or more protein domains, in its native 3-dimensional conformation. A native 3-dimensional conformation will comprise the levels of protein structure described above.

The biological function of any protein is dependent on its 3-dimensional conformation. Therefore, proper folding of a synthetic protein is required to enable its biological effects and properties. Suitable conditions must be applied to a polypeptide to ensure correct folding. Some proteins may fold correctly when suspended in aqueous solution and producing such proteins will not require additional steps comprising folding reactions. In some embodiments, the method does not comprise a step comprising a folding reaction. Some proteins require the formation of covalent disulfide bridges in order to achieve a native conformation, and producing such proteins will require additional steps comprising folding reactions. In some embodiments, the method comprises a step comprising a folding reaction. In order to form covalent disulfide bridges, which are important for the 3-dimensional structure of some proteins, the folding must be carried out in oxidative conditions.

Buffers for protein folding reactions typically comprise four key components: (i) and agent to aid in protein solubility (a chaotrope), (ii) an agent to buffer the pH of the reaction, (iii) an agent to facilitate formation and exchange of disulfide bridges (redox pair), (iv) a scavenger to prevent oxidation of methionine residues in the target protein, and (v) an acidification reagent added at the end to stop the folding reaction. Examples of component (i) include, but are not limited to, guanidine, urea, methanol, trifluoroethanol, and dimethyl sulfoxide (DMSO). Examples of component (ii) include, but are not limited to, Tris buffer, HEPES, and CHAPS. Examples of component (iii) include, but are not limited to, reduced and oxidized glutathione, and cysteine/cystine. An example of component (iv) includes, but is not limited to, methionine. Examples of component (v) include, but are not limited to, acetic acid, formic acid, and trifluoroacetic acid. An example of a folding reaction, using oxidative folding conditions for the folding of a plurality of structurally variant polypeptide molecules in parallel in the present invention is disclosed by way of a non-limiting example herein in Example 4.

The folding of polypeptides molecules can be performed in parallel in groups of, for example, 2, 4, 6, 8, 12, 24, 48, 96, 192, 288, 384, or more. Folding can be performed in parallel by applying folding conditions to the polypeptides of interest in parallel in suitable vessels. Such vessels include, but not limited to, 0.2 mL tubes, 0.5 mL tubes, 1.5 mL tubes, 2 mL tubes, 15 mL tubes, 48-well plates, 96-well plates, and 384-well plates.

In some embodiments of the present invention, the plurality of structurally variant polypeptide molecules is folded using uniform folding conditions applied in parallel.

In some embodiments of the present invention, the plurality of structurally variant polypeptide molecules is folded using uniform oxidative folding conditions applied in parallel.

Desalting and Lyophilization

The present invention provides a method for producing a plurality of structurally variant polypeptide molecules in which said plurality of structurally variant polypeptide molecules is desalted after being folded.

The folding of proteins requires polypeptides to be suspended in a solution providing suitable folding conditions. Components of the folding solution can inhibit downstream assays performed on the protein to determine its effects and properties. It is therefore useful to perform a desalting step to remove unwanted or harmful solvents and solutes. Unwanted or harmful components of the folding solution that are removed by desalting include, without limitation, chaotropes, alternative buffer compoinents, alternative redox pair components, alternative scavengers, acetic acid, alternative acidification agents, tris buffer, methionine, and redox pairs (such as oxidized and reduced glutathione).

As used herein, the term “desalting” is used to describe any technique used to separate a larger molecule of interest from smaller, unwanted salts, solutes, and chemicals contained in a solution or reaction mixture.

Desalting of polypeptides can be performed by a number of techniques known in the art. Column chromatography can be used for desalting. One form of column chromatography that can be used for desalting is specifically size exclusion chromatography in which the solution containing the molecule is passed through a solid phase of porous beads (Snyder 2000). Using size exclusion chromatography, the small solutes are slowed in their passage through the column due to their retention by the porous beads, while the larger desired molecules pass through quickly. Flushing the column with a chosen solvent ensures that the molecules exit the column dissolved in a desired final solvent. Another column chromatography technique is reversed phase chromatography. However, as discussed herein in column chromatography has numerous drawbacks in terms of time, cost, and scalability.

Desalting of polypeptides can be performed by a number of column-free techniques known in the art including, but not limited to, membrane filtration, column-free reverse-phase separation, and dialysis.

Desalting of polypeptides can be performed by membrane filtration techniques. In these techniques, the molecule of interest is retained by a membrane or a filter with a defined kDa cut-off, while unwanted solvents and solutes pass through and are discarded. The retained desired molecule can then be washed on the membrane or filter, and afterwards suspended in a desired solvent by the methods described above.

Examples of membrane filtration vessels that are suitable for desalting polypeptides include, but are not limited to, Microcon® Tubes (Millipore Sigma), Amicon Ultra Tubes (Millipore Sigma), and 96-well MultiScreen™ filter plates (Millipore Sigma).

The desalting of polypeptides by membrane filtration can be performed in parallel in groups of, for example, 2, 4, 6, 8, 12, 24, 48, 96, 192, 288, 384, or more. Desalting of polypeptides by membrane filtration can be performed in parallel by applying solutions containing the polypeptides of interest in parallel in suitable vessels. Such vessels include, but are not limited to, Microcon® Tubes (Millipore Sigma), Amicon Ultra Tubes (Millipore Sigma), and 96-well MultiScreen™ filter plates (Millipore Sigma).

Desalting of polypeptides can be performed by column-free reverse phase resin binding. In this technique, the solution containing the desired molecule is applied to a well or a container containing a hydrophobic resin that adsorbs the desired molecule. A suitable resin for use in desalting a polypeptide includes, but is not limited to, Chromabond® (Macherey-Nagel). With the desired molecule bound to the resin, the unwanted solvent and solutes can be removed, and the resin-bound molecules can be washed one or more times with a suitable washing solution. Suitable solutions for washing a resin-bound polypeptide include, but are not limited to, water, solution B (0.1% trifluoroacetic acid in 90% acetonitrile, 10% water, as described herein in Example 4).

The desalting of polypeptides by column-free reverse phase resin binding can be performed in parallel in groups of, for example, 2, 4, 6, 8, 12, 24, 48, 96, 192, 288, 384, or more. Desalting of polypeptides by column-free reverse phase resin binding can be performed in parallel by applying solutions containing the polypeptides of interest in parallel in suitable vessels. Such vessels include, but are not limited to, Chromabond 96-well plates (Macherey-Nagel), Sep-Pak C18 cartridges (Water), and Sep-Pak C18 96-well plates (Waters).

The use of Chromabond® resin for desalting a plurality of structurally variant polypeptide molecules in parallel in the present invention is disclosed by way of a non-limiting example herein in Example 4.

Desalting of polypeptides can be performed by dialysis techniques. Dialysis is a technique whereby the solution containing the molecule of interest (solution 1) is placed is a vessel with one or more porous surfaces. The pores of the vessel are of a size specified by kDa cut-off. The molecule of interest will be retained in the vessel because it is larger than the pores as defined by kDa cut-off. The vessel is then placed in a volume of a different, desired solution (solution 2). The undesired salts and solutes of solution 1 will pass out of the vessel through the pores by the process of osmosis and, likewise, the desired solution 2 will flow through the pores into the vessel. Thereby, the molecule of interest will be separated from solution 1 (e.g. the ligation reaction) by osmosis.

Suitable vessels for desalting polypeptides by dialysis include, but are not limited to, dialysis tubing, Slide-A-Lyzer™ dialysis casettes (ThermoFisher), Pur-A-Lyzer™ dialysis kits (Millipore Sigma), and Pierce™ 96-well Microdialysis plates (ThermoFisher).

Suitable kDa cut-offs for desalting polypeptides by dialysis include, but are not limited to, 3.5 kDa, 6 kDA, 8 kDa, 10 kDa, 12 kDa, 14 kDa, and 20 kDa.

The desalting of polypeptides by dialysis can be performed in parallel in groups of, for example, 2, 4, 6, 8, 12, 24, 48, 96, 192, 288, 384, or more. Desalting can be performed in parallel by applying solutions containing the polypeptides of interest in parallel in suitable dialysis vessels. Such vessels include, but are not limited to, dialysis tubing, Slide-A-Lyzer™ dialysis casettes (ThermoFisher), Pur-A-Lyzer™ dialysis kits (Millipore Sigma), and Pierce™ 96-well Microdialysis plates (ThermoFisher).

In some embodiments of the present invention, the plurality of folded structurally variant polypeptide molecules is desalted by membrane filtration.

In some embodiments of the present invention, the plurality of folded structurally variant polypeptide molecules is desalted by membrane filtration in parallel.

In some embodiments of the present invention, the plurality of folded structurally variant polypeptide molecules is desalted by column-free reverse phase resin binding.

In some embodiments of the present invention, the plurality of folded structurally variant polypeptide molecules is desalted by column-free reverse phase resin binding in parallel.

In some embodiments of the present invention, the plurality of folded structurally variant polypeptide molecules is desalted by dialysis.

In some embodiments of the present invention, the plurality of folded structurally variant polypeptide molecules is desalted by dialysis in parallel.

In some embodiments of the present invention, the plurality of folded structurally variant polypeptide molecules is lyophilized after desalting.

In some embodiments of the present invention, the plurality of folded structurally variant polypeptide molecules is lyophilized in parallel after desalting.

Lyophilization is the process whereby a molecule is completely dried, removing all traces of liquid solvent. The resulting lyophilized molecule is then present as a dry powder or crystal. Lyophilization can improve the stability of a molecule during prolonged storage. Lyophilization can also remove unwanted organic or inorganic solvents that might interfere with downstream uses of the molecule.

Lyophilization of polypeptides can be performed by, for example, freezing solutions containing the polypeptides of interest and then subjecting them to a vacuum until all of the solvent has sublimated. The lyophilization of plurality of structurally variant polypeptide molecules in parallel in the present invention is disclosed by way of a non-limiting example herein in Example 4.

Following lyophilisation, peptides can be resuspended in a solvent or buffer solution that is suitable for downstream analysis of their effects and properties. Suitable solvents include, but are not limited to, water, phosphate-buffered saline, cell culture media, dimethylsulfoxide, dimethylsulfoxide solution, ethanol solution, methanol solution, polyethylene glycol, polyethylene glycol solution, or any buffered aqueous solution compatible with assays on living cells and/or cell-free assays involving biomolecules.

The lyophilization of polypeptides can be performed in parallel in groups of, for example, 2, 4, 6, 8, 12, 24, 48, 96, 192, 288, 384, or more. Lyophilization can be performed in parallel by applying lyophilisation conditions as disclosed herein by non-limiting example in Example 4 to solutions containing the polypeptides of interest in parallel in suitable vessels. Such vessels include, but are not limited to, 0.2 mL tubes, 0.5 mL tubes, 1.5 mL tubes, 2 mL tubes, 15 mL tubes, 48-well plates, 96-well plates, and 384-well plates.

Evaluation and Analysis

The present invention provides a method for evaluating the effects and the properties of a plurality of structurally variant polypeptide molecules, said plurality of structurally variant polypeptide molecules having been produced by ligation, separation, folding, and desalting in parallel.

The present invention also provides a method for evaluating the effects and the properties of a plurality of structurally variant polypeptide molecules, said plurality of structurally variant polypeptide molecules having been produced by ligation, separation, folding, desalting, and lyophilization in parallel.

Lyophilized polypeptides can be resuspended in a solvent suitable for subsequent evaluation of their effects and/or properties. Suitable solvents for suspending lyophilized peptides for downstream analysis for use in the present invention include, but are not limited to, water, phosphate-buffered saline, cell culture media, dimethylsulfoxide, dimethylsulfoxide solution, ethanol solution, methanol solution, polyethylene glycol, and polyethylene glycol solution.

The effects of polypeptides on cells can be measured by contacting said polypeptides with cells. Polypeptides can be contacted with cells by mixing the polypeptide(s) of interest into the cell media, thereby providing the polypeptides in solution. Alternatively, the polypeptides can be adsorbed onto the surface of a cell culture plate, a well of a cell culture plate, or other suitable vessel to provide a polypeptide-coated surface for contacting cells. Polypeptides can be adsorbed onto the surface of vessels composed of certain materials including, but not limited to, polystyrene, polyvinylidene fluoride (PVDF), and mixed cellulose ester.

Cell culture vessels suitable for contacting cells with polypeptides of interest in parallel include, but are not limited to, 6-well plates, 12-well plates, 24-well plates, 48-well plates, 96-well plates, 128-well plates, 384-well plates, and cell culture dishes.

Once cells have been contacted with polypeptide(s) of interest, one of more effects of said polypeptide(s) on said cells can be evaluated by numerous assays known in the art. Suitable assays for use in the present invention include, but are not limited to, flow cytometry, polymerase chain reaction, real-time polymerase chain reaction, reverse-transcription polymerase chain reaction, western blot, enzyme-linked immunosorbent assay, enzyme-linked immunospot assay, cell migration assay, cell proliferation assay, cytotoxic killing assay, genome-wide sequencing, exome sequencing, RNA sequencing, chromatin immunoprecipitation sequencing, viral replication assay, fluorigenic or chromogenic reporter gene assay, FRET-or BRET-based reporter assays for protein-protein interactions, cell fusion assay, calcium flux assay, enzyme complementation assay, second messenger assay, receptor signaling assay, and cell surface antibody binding assay.

Useful effects of polypeptides on cells that can be evaluated by the present invention include, but are not limited to, binding to a ligand or receptor, blocking a ligand or receptor, stimulating cells, killing cells, and modulating cells. Desirable medical effects of a molecule include, but are not limited to, killing bacteria, disabling viruses, killing cancer cells, inhibiting cell proliferation, inhibiting disease pathways, and restoring the function of healthy pathways.

The use of a cell fusion assay to determine at least one effect of a plurality of structurally variant polypeptides on cells is disclosed as a non-limiting example herein in Example 5.

The use of a receptor signalling assay to determine at least one effect of a plurality of structurally variant polypeptides on cells is disclosed as a non-limiting example herein in Example 6.

The use of an assay to measure internalization of a receptor to determine at least one effect of a plurality of structurally variant polypeptides on cells is disclosed as a non-limiting example herein in Example 7.

The use of a bioluminescence resonance energy transfer (BRET) based reporter assay to measure at least one effect of a plurality of structurally variant polypeptides on cells is disclosed as a non-limiting example herein in Example 7

Cells that can be used for determining the effects and/or properties of polypeptides in the present invention include, but are not limited to, primary eukaryotic cells, transformed eukaryotic cells, immortal eukaryotic cells, cancer cells, ex vivo cells, and prokaryotic cells. In an embodiment, the cells are lymphocytes or leukocytes. In an embodiment, the cells are genetically modified cells that express target molecules that interact with the plurality of structurally variant polypeptides.

It is contemplated that in some embodiments of the present invention, polypeptides can be contacted with pathogens by any of the methods described above to measure the effects and/or properties of said polypeptides with respect to pathogens. Pathogens that could be contacted with polypeptides include, but are not limited to viruses such as HIV, HPV, MCV, influenza, Ebola, Measles; bacteria such as Staphylococcus, Enterococcus, Pseudomonas; and parasites such as Plasmodium, Toxoplasma, and Cryptosporidium.

The properties of polypeptides can be evaluated by techniques known in the art. Techniques that can be used in the present invention for evaluating the properties of polypeptides include, but are not limited to, radioligand binding assay, co-immunoprecipitation, bimolecular fluorescence complementation, affinity electrophoresis, label transfer, tandem affinity purification, proximity ligation assay, dual polarisation interferometry, static light scattering, dynamic light scattering, flow-induced dispersion analysis, ELISA, ELISPOT, surface plasmon resonance, precipitation titration, and protein array assay.

Useful properties of polypeptides for evaluation include, but are not limited to, improved stability, improved solubility, reduced toxicity, and increased or decreased binding to specific ligands or receptors

### Embodiments of the Present Invention

Non-limiting exemplary embodiments of the invention include:

1. A method for producing a plurality of folded structurally variant polypeptide molecules in parallel comprising:
a. providing a plurality of structurally variant regions of a polypeptide molecule in parallel;
b. ligating each of said plurality of structurally variant regions of a polypeptide molecule to a common, structurally invariant region of said polypeptide molecule in parallel separate ligation reactions to produce a plurality of structurally variant polypeptide molecules;
c. applying conditions to each of said separate ligation reactions in parallel to separate said plurality of structurally variant polypeptide molecules from each of said separate ligation reactions;
d. folding each of said plurality of structurally variant polypeptide molecules in parallel separate folding reactions to produce a plurality of folded structurally variant polypeptide molecules; and
e. applying conditions to each of said separate folding reactions in parallel to separate said plurality of folded structurally variant polypeptide molecules from said folding reactions.

2. The method according to embodiment 1, wherein providing a plurality of structurally variant regions of a polypeptide molecule in parallel is performed column-free.

3. The method according to any one of embodiments 1 or 2, wherein the ligation reactions comprise in-solution SEA native peptide ligations.

4. The method according to any one of embodiments 1 to 3, wherein the conditions applied to each of said separate ligation reactions in parallel comprise column-free separation.

5. The method according to embodiment 4, wherein the column-free separation comprises membrane filtration.

6. The method according to embodiment 4, wherein the column-free separation comprises precipitation/centrifugation.

7. The method according to embodiment 4, wherein the column-free separation comprises dialysis.

8. The method according to any one of embodiments 1 to 7, wherein the folding comprises uniform folding conditions.

9. The method according to embodiment 8, wherein the uniform folding conditions comprise oxidative folding.

10. The method according to any one of embodiments 1 to 9, wherein the conditions applied to each of said separate folding reactions in parallel comprise column-free separation.

11. The method according to embodiment 10, wherein the column-free separation comprises membrane filtration.

12. The method according to embodiment 10, wherein the column-free separation comprises reverse phase resin binding.

13. The method according to embodiment 10, wherein the column-free separation comprises dialysis.

14. The method according to any one of embodiments 1 to 13, wherein the plurality of folded structurally variant polypeptide molecules is lyophilized in parallel after step ‘e’.

15. The method according to embodiment 14, wherein the lyophilized plurality of folded structurally variant polypeptide molecules is suspended with a solvent after lyophilization.

16. The method according to embodiment 15, wherein the solvent is selected from the group comprising water, phosphate-buffered saline, cell culture media, dimethylsulfoxide, dimethylsulfoxide solution, ethanol solution, methanol solution, polyethylene glycol, and polyethylene glycol solution.

17. The method according to any one of embodiments 1 to 16, wherein all parallel steps are column-free.

18. A method for determining at least one effect of each of a plurality of folded structurally variant polypeptide molecules in parallel comprising:
a. providing a plurality of folded structurally variant polypeptide molecules by the method defined in any one of embodiments 1 to 18;
b. contacting the plurality of folded structurally variant polypeptide molecules separately in parallel with cells; and
c. determining at least one effect of each of the plurality of folded structurally variant polypeptide molecules on said cells.

19. The method according to embodiment 18, wherein the cells are selected from the group comprising bacteria, primary eukaryotic cells, transformed eukaryotic cells, and immortal eukaryotic cells.

20. The method according to any one of embodiments 18 or 19, wherein the at least one effect is determined by a method selected from the group comprising flow cytometry, polymerase chain reaction, real-time polymerase chain reaction, reverse-transcription polymerase chain reaction, western blot, enzyme-linked immunosorbent assay, enzyme-linked immunospot assay, cell migration assay, cell proliferation assay, cytotoxic killing assay, genome-wide sequencing, exome sequencing, RNA sequencing, chromatin immunoprecipitation sequencing, cell fusion assay, calcium flux assay, and cell surface antibody binding assay.

21. A method for determining at least one property of a plurality of folded structurally variant polypeptide molecules in parallel comprising:
a. providing a plurality of folded structurally variant polypeptide molecules by the method defined in any one of embodiments 1 to 17;
b. determining at least one property of each of the plurality of folded structurally variant polypeptide molecules.

22. The method according to embodiment 21, wherein the at least one property is determined by a method selected from the group comprising flow cytometry, polymerase chain reaction, real-time polymerase chain reaction, reverse-transcription polymerase chain reaction, western blot, enzyme-linked immunosorbent assay, enzyme-linked immunospot assay, cell migration assay, cell proliferation assay, cytotoxic killing assay, genome-wide sequencing, exome sequencing, RNA sequencing, chromatin immunoprecipitation sequencing, cell fusion assay, calcium flux assay, and cell surface antibody binding assay.

23. The method according to any one of embodiments 1 to 22, wherein the plurality of structurally variant regions of said polypeptide molecule are produced by solid phase peptide synthesis.

24. The method according to embodiment 23, wherein the solid phase peptide synthesis comprises Fmoc chemistry.

25. The method according to embodiment 23, wherein the solid phase peptide synthesis comprises Boc chemistry.

26. The method according to any one of embodiments 23 to 25, wherein the plurality of structurally variant regions of said polypeptide molecule are produced in parallel by solid phase peptide synthesis in multi-well plates using a parallel peptide synthesizer.

27. The method according to any one of embodiments 1 to 26, wherein the common, structurally invariant region of said polypeptide molecule is produced by solid phase peptide synthesis.

28. The method according to embodiment 27, wherein the solid phase peptide synthesis comprises Fmoc chemistry.

29. The method according to embodiment 27, wherein the solid phase peptide synthesis comprises Boc chemistry.

30. The method according to any one of embodiments 1 to 29, wherein the structurally variant regions of said polypeptide molecule are tag-free.

31. The method according to any one of embodiments 1 to 30, wherein the common, structurally invariant region of said polypeptide molecule is tag-free.

32. The method according to any one of embodiments 1 to 31, wherein the structurally variant regions of said polypeptide molecule comprise amino acid analogs.

33. The method according to any one of embodiments 1 to 32, wherein the common, structurally invariant region of said polypeptide molecule comprises amino acid analogs.

34. The method according to any one of embodiments 1 to 33, wherein the plurality of folded structurally variant polypeptide molecules are proteins.

35. The method according to embodiment 34, wherein the proteins are protein analogs.

36. The method according to embodiment 34, wherein the proteins are cytokines.

37. The method according to embodiment 36, wherein the cytokines are cytokine analogs.

38. The method according to embodiment 34, wherein the proteins are chemokines.

39. The method according to embodiment 38, wherein the chemokines are chemokine analogs.

40. A method for producing a plurality of structurally variant polypeptide molecules in parallel comprising:
a. providing a plurality of structurally variant regions of a polypeptide molecule in parallel;
b. ligating each of said plurality of structurally variant regions of a polypeptide molecule to a common, structurally invariant region of said polypeptide molecule in parallel separate ligation reactions to produce a plurality of structurally variant polypeptide molecules; and
c. applying conditions to each of said separate ligation reactions in parallel to separate said plurality of structurally variant polypeptide molecules from each of said separate ligation reactions.

41. The method of embodiment 40, wherein after step the method further comprises:
d. folding each of said plurality of structurally variant polypeptide molecules in parallel separate folding reactions to produce a plurality of folded structurally variant polypeptide molecules; and
e. applying conditions to each of said separate folding reactions in parallel to separate said plurality of folded structurally variant polypeptide molecules from said folding reactions.

42. The method according to embodiment 40 or 41, wherein providing a plurality of structurally variant regions of a polypeptide molecule in parallel is performed column-free.

43. The method according to any one of embodiments 40 to 42, wherein the ligation reactions comprise in-solution SEA native peptide ligations.

44. The method according to any one of embodiments 40 to 43, wherein the conditions applied to each of said separate ligation reactions in parallel comprise column-free separation.

45. The method according to embodiment 44, wherein the column-free separation comprises membrane filtration.

46. The method according to embodiment 44, wherein the column-free separation comprises precipitation/centrifugation.

47. The method according to embodiment 44, wherein the column-free separation comprises dialysis.

48. The method according to any one of embodiments 41 to 47, wherein the folding comprises uniform folding conditions.

49. The method according to embodiment 48, wherein the uniform folding conditions comprise oxidative folding.

50. The method according to any one of embodiments 41 to 49, wherein the conditions applied to each of said separate folding reactions in parallel comprise column-free separation.

51. The method according to embodiment 50, wherein the column-free separation comprises membrane filtration.

52. The method according to embodiment 50, wherein the column-free separation comprises reverse phase resin binding.

53. The method according to embodiment 50, wherein the column-free separation comprises dialysis.

54. The method according to any one of embodiments 41 to 53, wherein the plurality of folded structurally variant polypeptide molecules is lyophilized in parallel after step ce'.

55. The method according to any one of embodiments 40 or 42 to 47, wherein the plurality of structurally variant polypeptide molecules is lyophilized in parallel after step ‘c’.

56. The method according to embodiment 54 or 55, wherein the lyophilized polypeptide molecules are suspended with a solvent after lyophilization.

57. The method according to embodiment 56, wherein the solvent is selected from the group comprising water, phosphate-buffered saline, cell culture media, dimethylsulfoxide, dimethylsulfoxide solution, ethanol solution, methanol solution, polyethylene glycol, and polyethylene glycol solution.

58. The method according to any one of embodiments 1 to 57, wherein all parallel steps are column-free.

59. A method for determining at least one effect of each of a plurality of structurally variant polypeptide molecules in parallel comprising:
a. providing a plurality of structurally variant polypeptide molecules by the method defined in any one of embodiments 40, 42 to 47, and 55 to 58;
b. contacting the plurality of structurally variant polypeptide molecules separately in parallel with cells; and
c. determining at least one effect of each of the plurality of structurally variant polypeptide molecules on said cells.

60. A method for determining at least one effect of each of a plurality of folded structurally variant polypeptide molecules in parallel comprising:
a. providing a plurality of folded structurally variant polypeptide molecules by the method defined in any one of embodiments 41 to 58;
b. contacting the plurality of folded structurally variant polypeptide molecules separately in parallel with cells; and
c. determining at least one effect of each of the plurality of folded structurally variant polypeptide molecules on said cells.

61. The method according to embodiment 59 or 60, wherein the cells are selected from the group comprising bacteria, genetically modified eukaryotic cells, primary eukaryotic cells, transformed eukaryotic cells, and immortal eukaryotic cells.

62. The method according to any one of embodiments 59 to 61, wherein the at least one effect is determined by a method selected from the group comprising flow cytometry, polymerase chain reaction, real-time polymerase chain reaction, reverse-transcription polymerase chain reaction, western blot, enzyme-linked immunosorbent assay, enzyme-linked immunospot assay, cell migration assay, cell proliferation assay, cytotoxic killing assay, genome-wide sequencing, exome sequencing, RNA sequencing, chromatin immunoprecipitation sequencing, cell fusion assay, calcium flux assay, and cell surface antibody binding assay.

63. A method for determining at least one property of a plurality of structurally variant polypeptide molecules in parallel comprising:
a. providing a plurality of structurally variant polypeptide molecules by the method defined in any one of embodiments 40, 42 to 47, and 55 to 58;
b. determining at least one property of each of the plurality of structurally variant polypeptide molecules.

64. A method for determining at least one property of a plurality of folded structurally variant polypeptide molecules in parallel comprising:
a. providing a plurality of folded structurally variant polypeptide molecules by the method defined in any one of embodiments 41 to 58;
b. determining at least one property of each of the plurality of folded structurally variant polypeptide molecules.

65. The method according to embodiment 63 or 64, wherein the at least one property is determined by a method selected from the group comprising flow cytometry, polymerase chain reaction, real-time polymerase chain reaction, reverse-transcription polymerase chain reaction, western blot, enzyme-linked immunosorbent assay, enzyme-linked immunospot assay, cell migration assay, cell proliferation assay, cytotoxic killing assay, genome-wide sequencing, exome sequencing, RNA sequencing, chromatin immunoprecipitation sequencing, cell fusion assay, calcium flux assay, and cell surface antibody binding assay.

66. The method according to any one of embodiments 40 to 65, wherein the plurality of structurally variant regions of said polypeptide molecule are produced by solid phase peptide synthesis.

67. The method according to embodiment 66, wherein the solid phase peptide synthesis comprises Fmoc chemistry.

68. The method according to embodiment 66, wherein the solid phase peptide synthesis comprises Boc chemistry.

69. The method according to any one of embodiments 66 to 68, wherein the plurality of structurally variant regions of said polypeptide molecule are produced in parallel by solid phase peptide synthesis in multi-well plates using a parallel peptide synthesizer.

70. The method according to any one of embodiments 40 to 69, wherein the common, structurally invariant region of said polypeptide molecule is produced by solid phase peptide synthesis.

71. The method according to embodiment 70, wherein the solid phase peptide synthesis comprises Fmoc chemistry.

72. The method according to embodiment 70, wherein the solid phase peptide synthesis comprises Boc chemistry.

73. The method according to any one of embodiments 40 to 72, wherein the structurally variant regions of said polypeptide molecule are tag-free.

74. The method according to any one of embodiments 40 to 73, wherein the common, structurally invariant region of said polypeptide molecule is tag-free.

75. The method according to any one of embodiments 40 to 74, wherein the structurally variant regions of said polypeptide molecule comprise amino acid analogs.

76. The method according to any one of embodiments 40 to 75, wherein the common, structurally invariant region of said polypeptide molecule comprises amino acid analogs.

77. The method according to any one of embodiments 40 to 76, wherein the plurality of folded structurally variant polypeptide molecules are proteins.

78. The method according to any one of embodiments 40, 42 to 47, 55 to 59, 61 to 63, or 65 to 76, wherein the plurality of structurally variant polypeptide molecules are proteins.

79. The method according to embodiment 77 or 78, wherein the plurality of structurally variant regions of said polypeptide molecule corresponds to a region of a protein, and wherein the common, structurally invariant region of said polypeptide molecule corresponds to a region of the same protein.

80. The method according to embodiment 77 or 78, wherein the plurality of structurally variant regions of said polypeptide molecule corresponds to a region of a first protein, and wherein the common, structurally invariant region of said polypeptide molecule corresponds to a region of a second protein.

81. The method according to embodiment 77 or 78, wherein the plurality of structurally variant regions of said polypeptide molecule are artificial polypeptides, and wherein the common, structurally invariant region of said polypeptide molecule corresponds to a region of a protein.

82. The method according to embodiment 77 or 78, wherein the plurality of structurally variant regions of said polypeptide molecule corresponds to a region of a protein, and wherein the common, structurally invariant region of said polypeptide molecule is an artificial polypeptide.

83. The method according to any one of embodiments 77 to 82, wherein the proteins are protein analogs.

84. The method according to embodiment 77 to 82, wherein the proteins are cytokines.

85. The method according to embodiment 84, wherein the cytokines are cytokine analogs.

86. The method according to embodiment 77 to 82, wherein the proteins are chemokines.

87. The method according to embodiment 86, wherein the chemokines are chemokine analogs.

The invention is further illustrated by the following non-limiting examples.

### EXAMPLES

### Example 1—Batch Synthesis of the Invariant Core Fragment of RANTES/CCL5

To provide the common, structurally invariant region of RANTES/CCL5, large batches of the C-terminal fragment, RANTES(11-68), were prepared by solid-phase peptide synthesis. RANTES(11-68) constituted the Core Fragment, unchanged across the panel of analogs, that was used for downstream parallel ligation reactions with a plurality of N-terminal structurally variant regions of RANTES/CCL5.

Two batches of the core chemokine fragment RANTES(11-68) were prepared. Core Fragment Batch 1 was prepared by synthesizing a RANTES/CCL5(34-68) fragment using Boc chemistry on a ABI 433 peptide synthesizer, and synthesizing a RANTES/CCL5(11-33)-C-terminal thioester fragment using Fmoc chemistry on a Prelude® synthesizer. RANTES/CCL5(34-68) fragment and RANTES/CCL5(11-33)-C-terminal thioester fragment were then joined by native chemical ligation to produce RANTES(11-68) Core Fragment Batch 1. Core Fragment Batch 2 was prepared by synthesizing the full-length fragment using Fmoc chemistry on a Prelude® synthesizer. After synthesis, both batches were purified by reverse-phase HPLC (RP-HPLC) using a Waters1525 system with a Vydac® 250×22 mm C8 column, and subjected to MALDI MS analysis on an AB Sciex 4800 MALDI TOF/TOF™ mass spectrometer (linear positive mode, using 2,5-dihydroxybenzoic acid as matrix).

MALDI MS analysis of Core Fragment Batch 1 revealed a mass consistent with that of the target product (expected mass 6812 Da, observed mass 6806 Da). MALDI MS analysis of Core Fragment Batch 2 revealed a mass (6832 Da) consistent with the target product carrying an oxidized methionine residue (expected mass 6828 Da). Complete oxidation of Met⁶⁷ to Met⁶⁷(O) in this fragment was confirmed by MALDI MS analysis of tryptic peptides.

### Example 2—Parallel Plate-Based Synthesis of N-terminal Structurally Variant Regions of RANTES/CCL5

To provide a plurality of structurally variant regions of RANTES/CCL5, 96 N-terminal-SEA peptides, corresponding to residues 0-10 of a group of 96 RANTES/CCL5 analogs were synthesized in parallel in individual wells on a parallel synthesis plate. These peptides constituted the Variant Regions that were used for downstream parallel ligation reactions with C-terminal Core Fragments of RANTES/CCL5.

Fragments corresponding to N-terminal residues 0-10 of a set of previously identified RANTES/CCL5 analogs (Gaertner 2008) were synthesized at 2 pmol scale on an Intavis MultiPep RSi 384-well peptide synthesizer using bis(2-sulfanylethyl)amino (SEA) resin prepared according to previously described methods (Ollivier 2010) so that cleavage would yield fragments in the C-terminal thioester format required for the in-well native chemical ligation step. After resin cleavage, the crude product in each well was dissolved in 500 μL water/acetonitrile (1:1) containing 1% TFA. A volume of this solution corresponding to an estimated 0.6 μmol peptide (150 μL) was transferred to a 2 mL deep well 96-well polypropylene plate and lyophilized. The Variant Region peptides were provided in parallel, column-free.

87 of the 96 reactions yielded products corresponding to peptides with the expected masses, with nine syntheses yielding products corresponding to capped truncated peptides (Table 1).

### Example 3—Parallel In-well Ligation of Variant Regions to Core Fragment of RANTES/CCL5 and Size Exclusion

To produce a plurality of complete, variant RANTES/CCL5 analogs, in-well native chemical ligation reactions between C-terminal Core Fragment [RANTES/CCL5(11-68)] produced as described in Example 1 with each of the Variant Region N-terminal SEA-thioester peptides [RANTES/CCLS(0-10)] produced as described in Example 2 were performed in parallel in a deep well 96-well polypropylene plate. 51 ligations used Core Fragment Batch 1 and 36 ligations used Core Fragment Batch 2 to produce 87 RANTES/CCL5 analogs in total.

In-well native chemical ligation was carried out using an estimated six-fold excess (0.6 μmol) of the Variant Region N-terminal SEA fragment over the C-terminal Core Fragment (0.1 μmol) in each of the 87 reactions, in parallel. A 1 mM solution of Core Fragment was prepared in ligation buffer (0.2 M sodium phosphate buffer, pH 7.2, containing 6 M guanidine hydrochloride, 50 mM Methionine, 0.1 M 4-mercaptophenylacetic acid and 0.1 M tris(2-carboxyethyl)phosphine), and 100 μL of this solution was added to each well containing lyophilized crude Variant Region N-terminal SEA fragment synthesis product. The plate was then sealed and the reaction mixtures were stirred overnight at 37° C.

Following ligation, excess unreacted N-terminal peptide and the other constituents of the ligation buffer, including thiol scavengers, were removed using a parallel size exclusion step. Ligation mixtures from wells were applied to Millipore Microcon® tubes with 10 kDa cut-off membranes which had been prewet with a solution of 6M guanidine hydrochloride. The tubes were then centrifuged 10 min at 14000×g and the flow through was discarded. Three washing steps were carried by applying 150 pL 6M guanidine hydrochloride solution and centrifuging 10 min at 14000×g, then retentates were supplemented with 50 μL of a solution of 0.28 M tris(2-carboxyethyl)phosphine dissolved in 6 M guanidine hydrochloride, pH 5.3 and left at ambient temperature for 30 min without agitation. Eight further 6 M guanidine hydrochloride solution washing steps were carried out, then the retentates (100 μL) were recovered by inverting the Microcon® tube inserts and placing them above receiving tubes provided by the manufacturer, then centrifuging at 1000×g for 4 min.

### Example 4—Parallel Folding, Desalting, and Lyophilization of Ligated RANTES/CCL5 Polypeptide Analogs

To produce a plurality of folded RANTES/CCL5 analogs, 85 ligated and size-excluded RANTES/CCL5 polypeptide analogs produced as described in Example 3 were subjected to an in-well folding step and a final in-well desalting step in parallel in a deep well 96-well plate.

Folding of the ligated material was performed by adding 1.2 mL of folding buffer (2 M guanidine hydrochloride, 0.1 M Tris base, 0.5 mM reduced glutathione, 0.3 mM oxidized glutathione, 10mM methionine, pH 8.0) directly to each RANTES/CCL5 analog in parallel in a deep well 96-well plate, then leaving the mixtures at ambient temperature for three days without agitation.

For desalting, the folding reactions were acidified by adding to each of the folding reactions 50 μL acetic acid (33% v/v) in parallel, with each reaction then divided into three 45 μL aliquots and placed in wells of a 2 mL deep well 96-well polypropylene plate. 900 μL of 2 M guanidine hydrochloride was added to each well in parallel, and the well contents were transferred into wells of fritted 96-well plates that had been filled with C18 Chromabond® resin (Machery-Nagel, 130 mg per well), pretreated with 500 μL acetonitrile and equilibrated with two washes (500 μL) of 5% Solvent B (0.1% trifluoroacetic acid in 90% acetonitrile, 10% water), 95% Solvent A (0.1% trifluoroacetic acid in water). Resin in wells was washed four times using 500 μL of a 5% Solvent B and eluted into recovery deep well plates using two 200 μL volumes of a mixture 50% Solvent B followed by one 200 μL volume of 90% Solvent B.

After desalting the eluates were lyophilized. Eluates were frozen overnight in deep well 96-well plates, the plates were placed in a speedvac™ rotor (Savant™ SVC 200, ThermoFisher) compatible with deep well plates, and spun at 2000 g overnight in a vacuum.

In order to characterize the final products (the plurality of variant, folded RANTES/CCL5 analogs), the lyophilized RANTES/CCL5 analogs were dissolved in 250 μL water, with 0.5 μL samples taken for MALDI MS analysis on an AB Sciex 4800 MALDI TOF/TOF™ mass spectrometer (linear positive mode, using 2,5-dihydroxybenzoic acid as matrix), and 2.5 μL samples taken for RP-HPLC analysis using an Alliance 2695 system (Waters) and a nucleosil® C8-300-5 column (Machery Nagel), with a gradient of 10% to 70% Solvent B/Solvent A at 1% per minute.

Despite the absence of any chromatography purification steps, MALDI MS analysis revealed that the parallel in-well ligation and folding reactions all yielded products with (i) a single observed mass corresponding to that of the folded target product (reactions using Core Fragment Batch 1, Table 2A), (ii) a single observed mass corresponding to that of the folded target product incorporating Met⁶⁷(O) (a subset of the reactions using Core Fragment Batch 2, Table 2B), or (iii) two observed masses, one corresponding to that of the folded target product, the other corresponding to that of the folded target product incorporating Met⁶⁷(O) (the remainder of reactions using Core Fragment Batch 2, Table 2B).

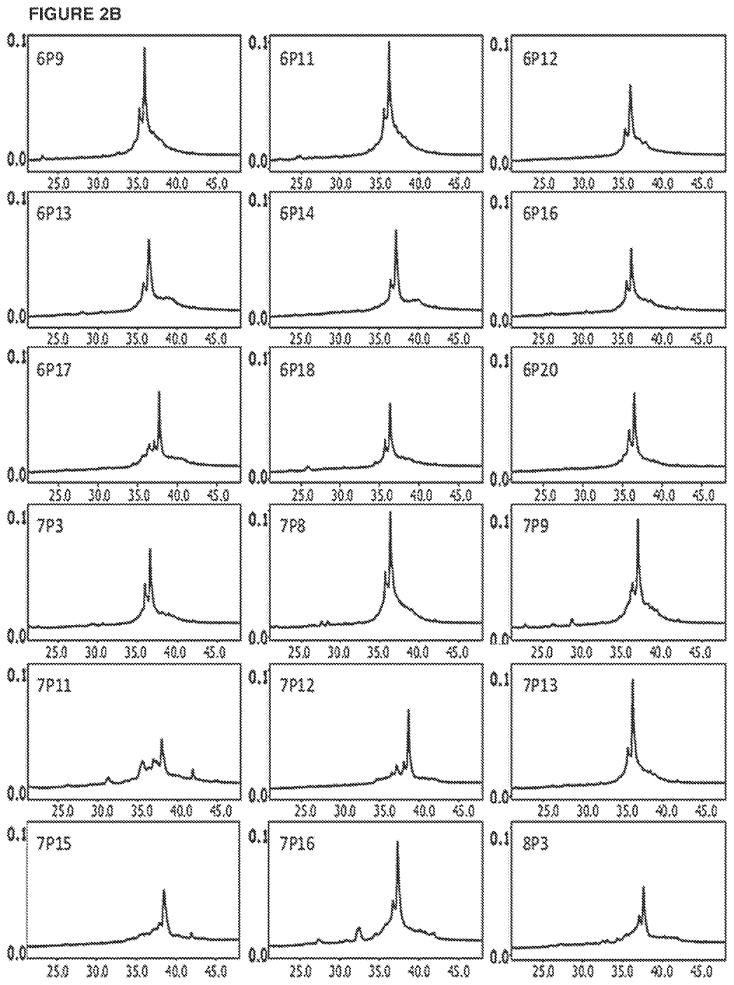

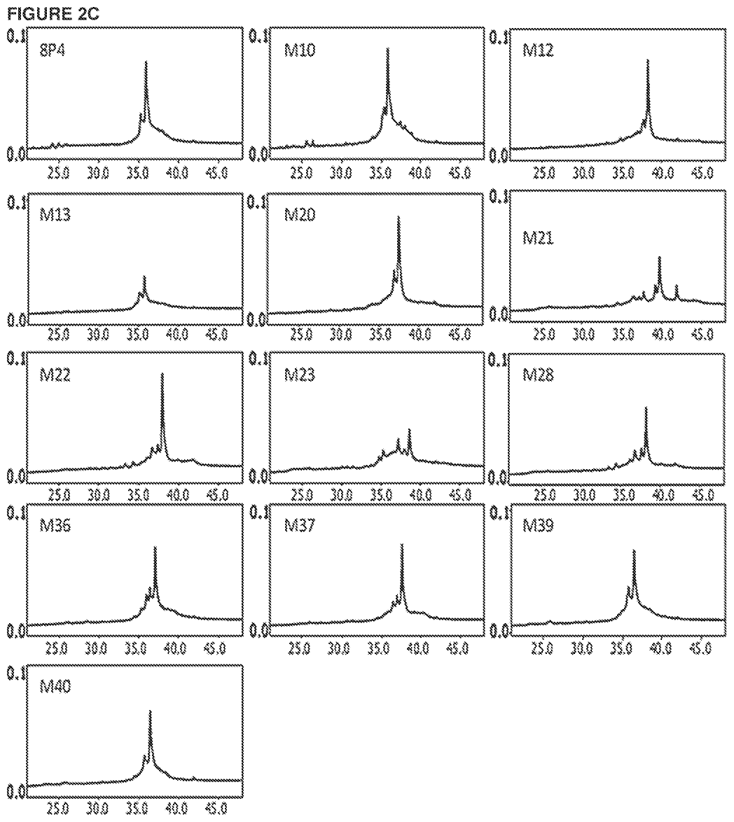

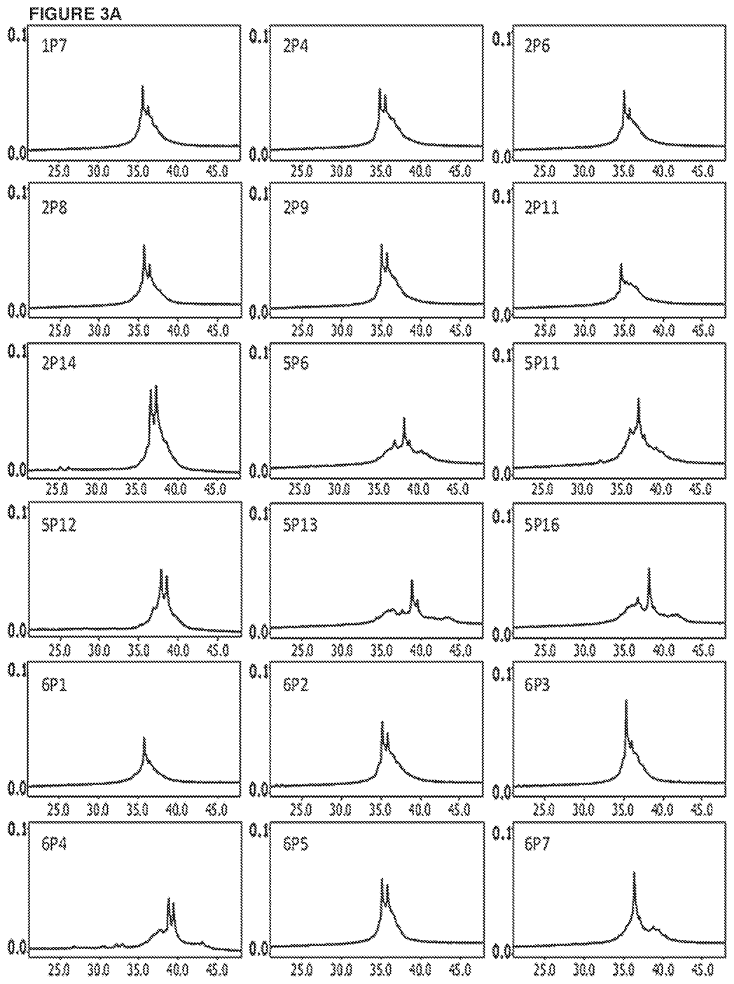

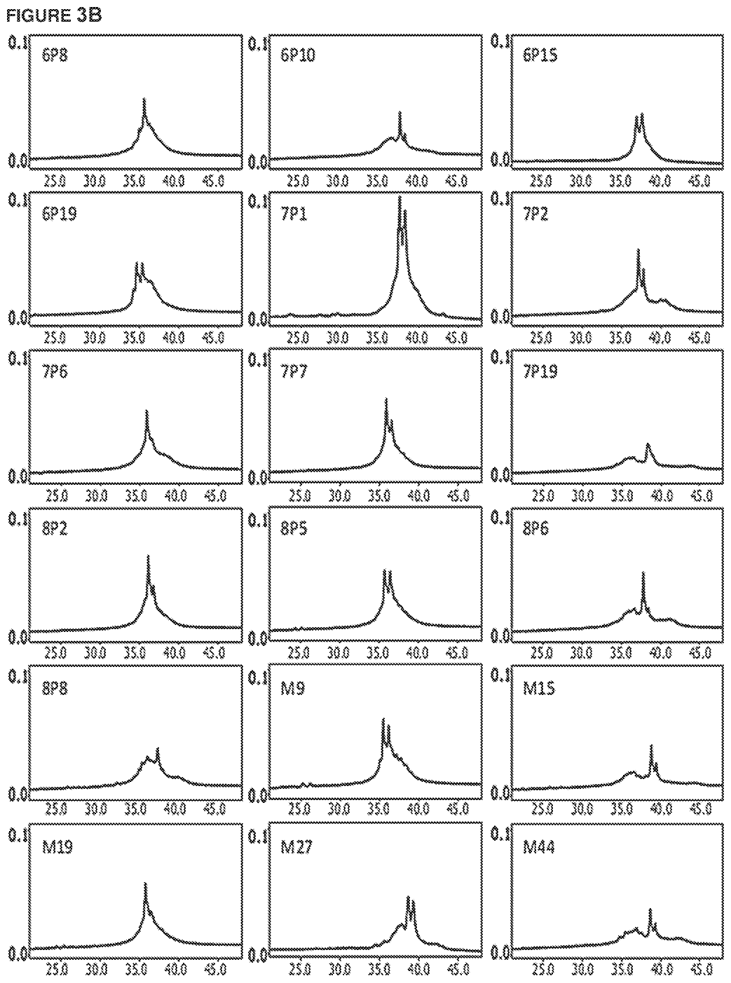

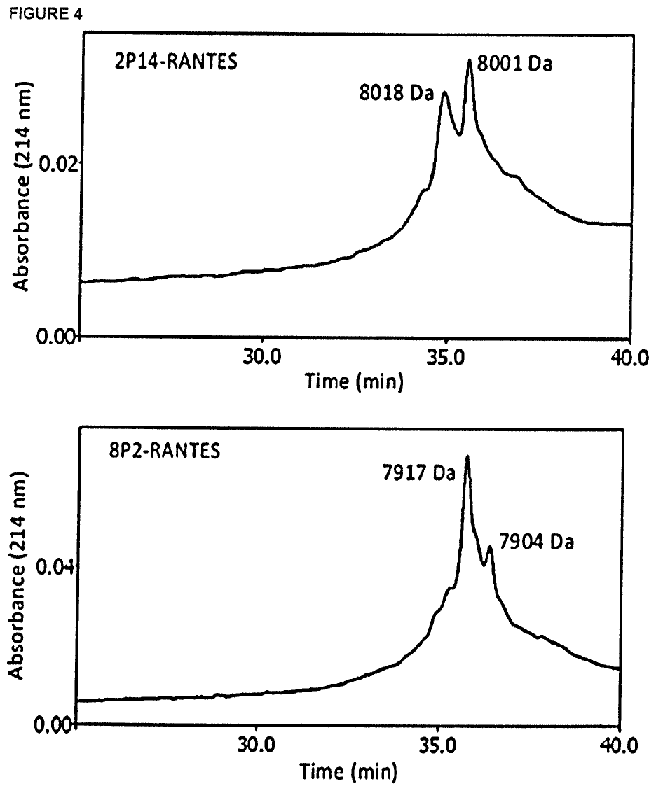

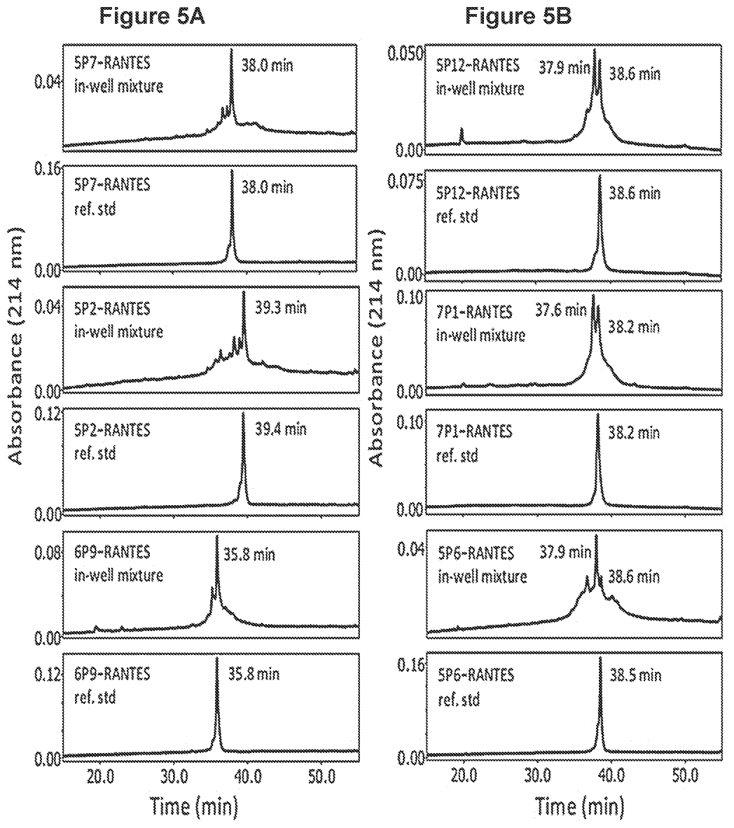

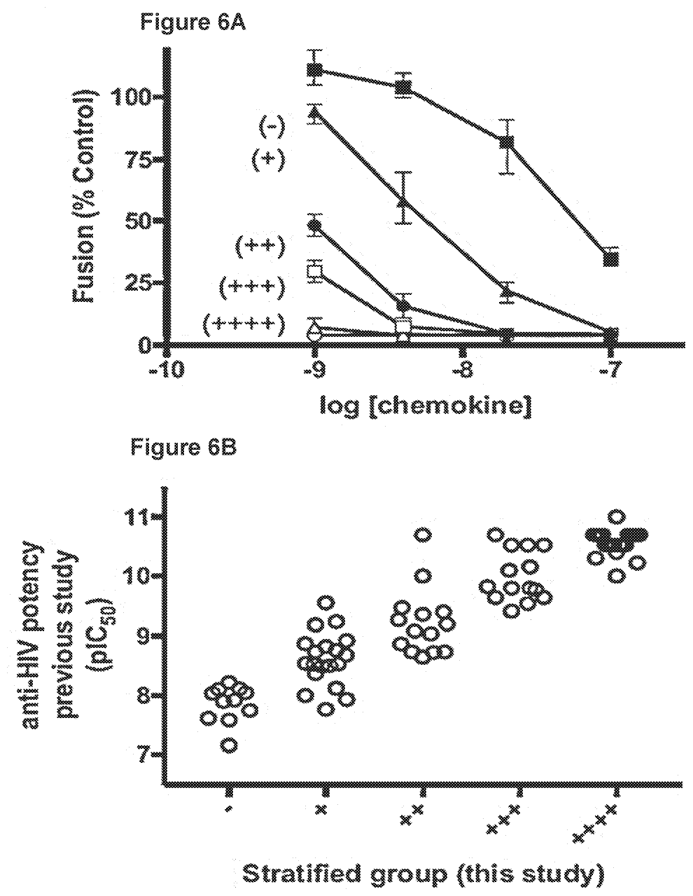

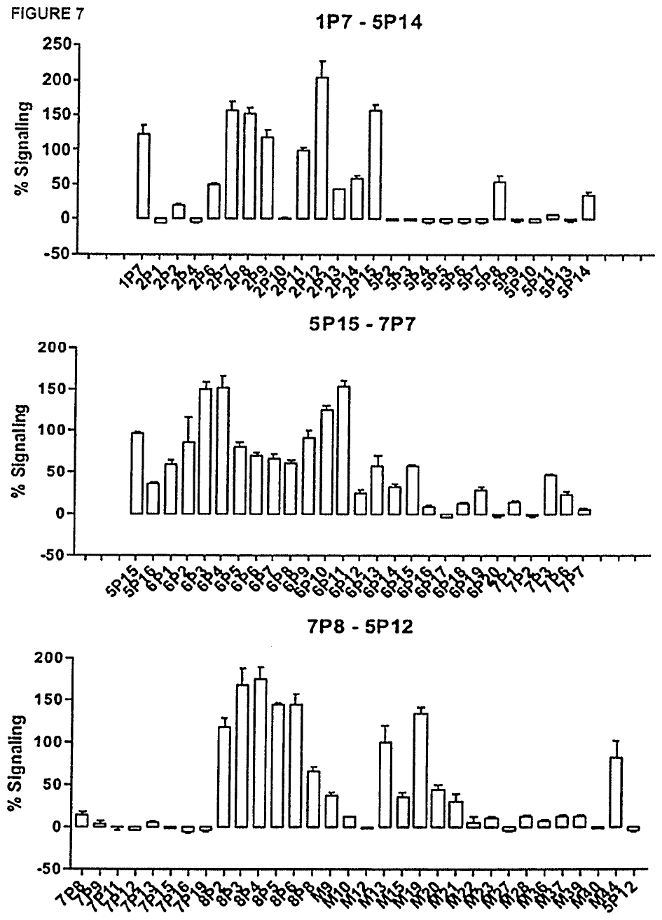

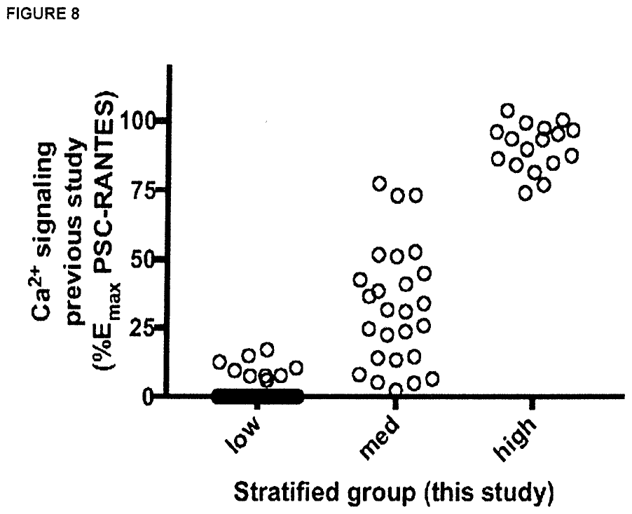

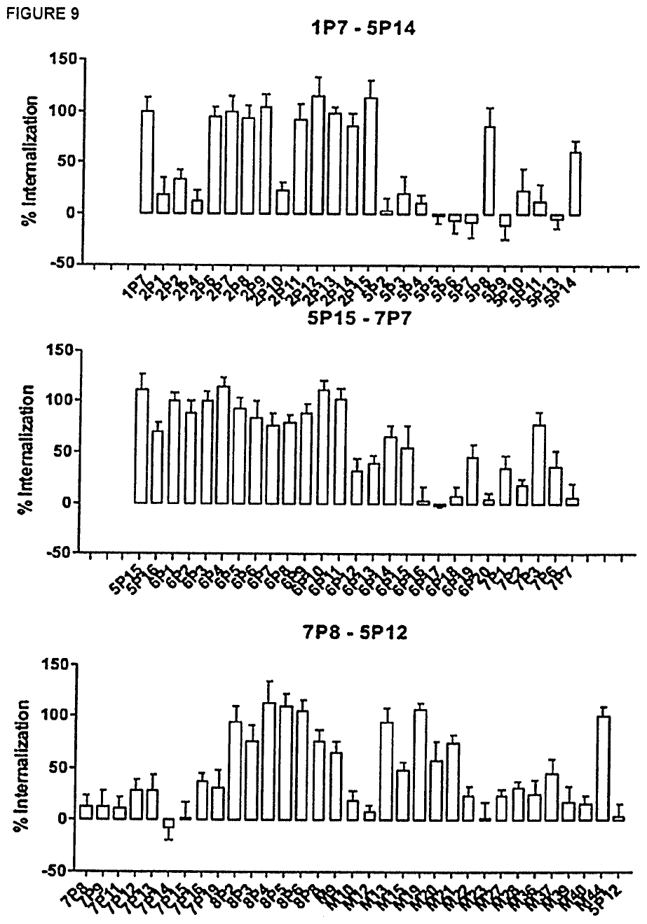

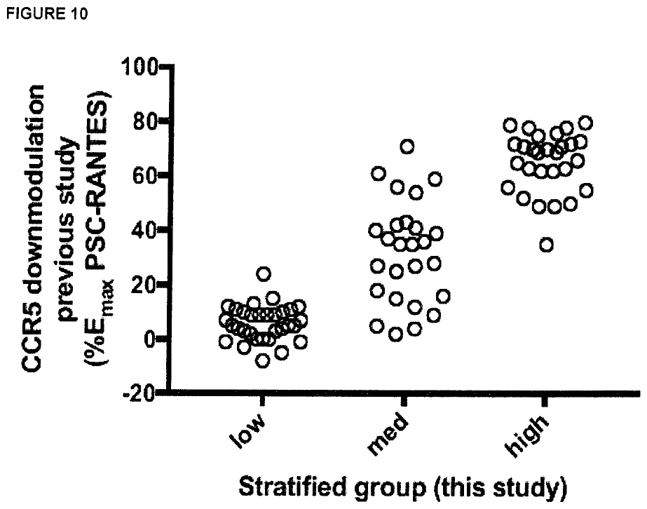

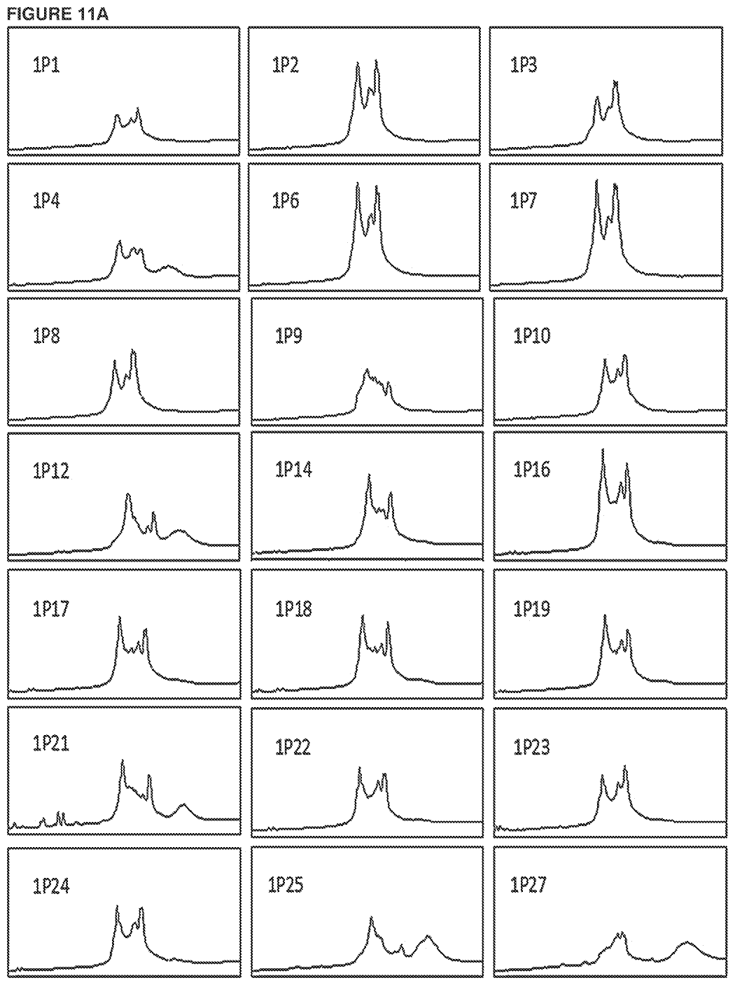

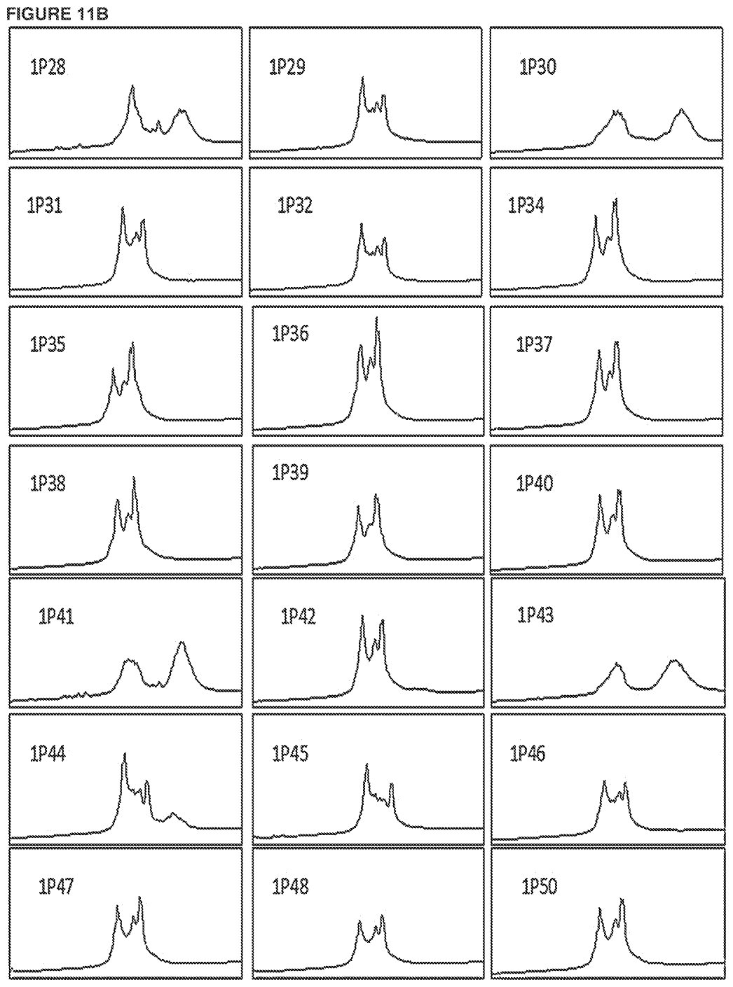

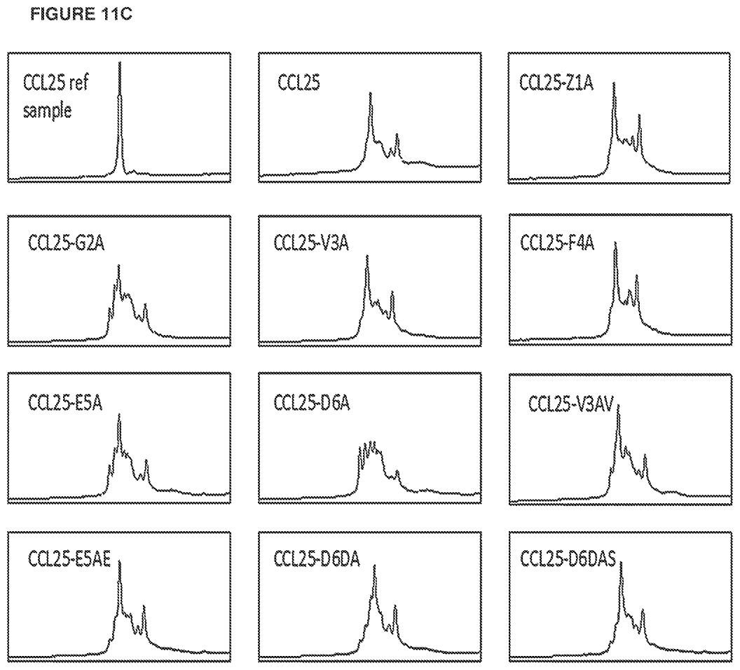

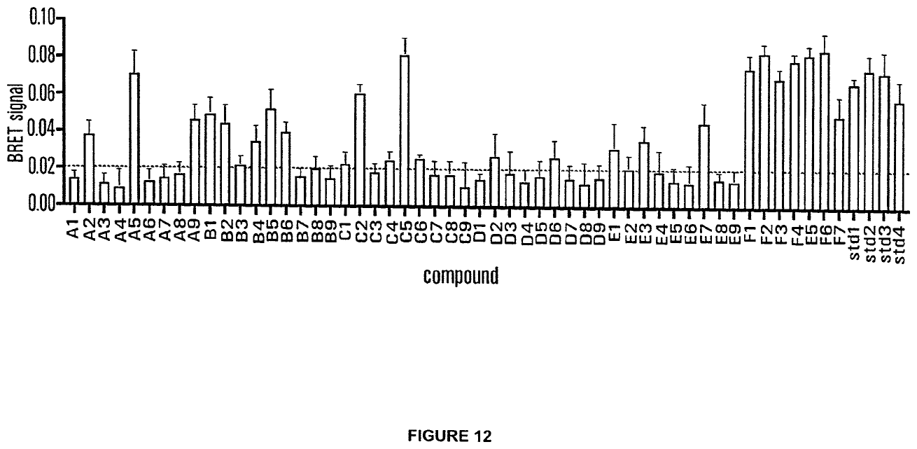

Similarly, analysis by RP-HPLC revealed either a single major peak (reactions using Core Fragment Batch 1, FIGS. 2, 2A, and 2B), or two major peaks (reactions using Core Fragment Batch 2, e.g. 2P14-RANTES, 8P2-RANTES, FIGS. 3 and 3A). Further analysis of the double major peaks in two representative wells (2P14-RANTES and 8P2-RANTES) showed that in both cases, the peak with the longer retention time has a mass consistent with that of the target protein, and the peak with the shorter retention time has a mass consistent with that of the target protein incorporating a single oxidized methionine, Met⁶⁷(O) (FIG. 4). The product with non-oxidized Met⁶⁷ derived from fully Met⁶⁷(O) Core Fragment Batch 2 in these syntheses was presumably the consequence of partial reduction of the oxidized Met⁶⁷(O) residue under the reducing conditions used for the ligation reaction.

Six selected wells, representing syntheses of differing quality in the preliminary analysis (5P12-RANTES, 7P1-RANTES, 5P6-RANTES 5P7-RANTES, 5P2-RANTES and 6P9-RANTES), were subjected to further analysis by RP-HPLC. Retention times of the major peaks were compared with those of corresponding reference standard chemokines (FIG. 5). For the syntheses using Core Fragment Batch 1, the single major peak in each case had an elution profile consistent with that of the reference standard sample. For the syntheses using Core Fragment Batch 2, in the cases where double major peaks were apparent (5P12-RANTES and 7P1-RANTES), the peaks with the longer retention times had elution profiles consistent with those of the reference standard samples. In the case where only a single major peak was apparent (5P6-RANTES), its retention time was not consistent with that of the reference standard sample, but had the reduced retention time characteristic of a Met⁶⁷(O) variant. A shoulder peak showed a retention time consistent with that of the reference standard sample, indicating that the non-oxidized Met⁶⁷ variant may have been present as a minority product.

To estimate the range of yields in this parallel synthesis, the six selected wells (5P12-RANTES, 7P1-RANTES, 5P6-RANTES 5P7-RANTES, 5P2-RANTES and 6P9-RANTES) which included syntheses providing both high yield (e.g. 7P1-RANTES, 5P7-RANTES) and lower yields (e.g. 5P6-RANTES, 5P2-RANTES), were analyzed using HPLC analysis software to estimate percentage purity, based on peak area, of the peaks corresponding to the target product. Since modifications at the C-terminus of RANTES/CCLS do not affect pharmacological activity (Escola 2010), the peaks corresponding to the Met⁶⁷(O) congener were considered as part of the total target product yield when estimating final yields per well. For the six wells, we also estimated total protein content by dissolving the contents of the well in 250 μL water and measuring absorbance at 280 nm, making use of the predicted extinction coefficients of the analogs.

The in-well ligation and folding procedure purities in the group of six wells provided target protein purities spanning the range 17-56%, corresponding to yields of approximately 7-14% with respect to the C-terminal target fragment. For well contents dissolved in 250 μL water, the estimated concentration of target protein ranged from 26-56 μM (Table 3). A nominal concentration of 50 μM was defined for each target protein, noting that this concentration was likely to be an overestimate for certain well mixtures whose analytical RP-HPLC traces (FIGS. 2, 2A, and 2B) indicated the lowest levels of purity and yield (e.g. M44-RANTES, 7P19-RANTES, M23-RANTES).

### Example 5—Evaluating Anti-HIV Potency of RANTES/CCL5 Analogs by Cell Fusion Assay

The pharmacological activity of RANTES/CCL5 analogs as produced by the method described in Example 4 was determined using an R5-dependent envelope mediate cell fusion assay. R5-tropic envelope-dependent cell-fusion assays were carried out as previously described (Hartley 2004; Gaertner 2008; Cerini 2008) using HeLa-P5L (Simmons 1997) and HeLa-Env-ADA (Pleskoff 1997) cell lines.

Each chemokine analog was tested for anti-HIV potency using a cell fusion assay, scoring each compound for its capacity to block cell fusion at each of four estimated concentrations: 1 nM, 4.6 nM, 21.5 nM and 100 nM. The compounds were divided into five groups: 1; complete inhibition not achieved at any concentration, 2; complete inhibition only achieved at the highest concentration (100 nM), 3; complete inhibition achieved at the two highest concentrations (21.5 nM and 100 nM), 4; complete inhibition achieved at three concentrations (4.6nM, 21.5 nM and 100 nM), and 5; complete inhibition achieved at all four concentrations (FIG. 6A). When the parallel-synthesized analogs were divided into anti-HIV potency groups in this way and compared with the pIC50 values obtained using the corresponding reference standard chemokine analogs in an earlier study (Gaertner 2008), a good correlation was obtained (FIG. 6B), with analogs in Groups 1 to 5 corresponding to p1050 values from the original study spanning the ranges 7.2-8.2, 7.8-9.6, 8.6-10.0, 9.4-10.7 and 10.0-11.0, respectively. This indicates that screening parallel-synthesized chemokine analogs produced by the method described in Example 4 is sufficient to identify the most potent anti-HIV chemokine analogs from a panel, as well as to stratify the less potent analogs with a reasonably high resolution.

### Example 6—Evaluating RANTES/CCL5 Analogs by CCR5 Agonist Assay

The pharmacological activity of RANTES/CCL5 analogs as produced by the method described in Example 4 was determined by measuring CCR5 agonist activity, an undesirable characteristic from a safety perspective, using a calcium flux assay. For this assay, HEK-CCR5 cells were used. A stably transduced clonal human embryonic kidney 293 (HEK) cell line was obtained transduction with a lentiviral vector (Hartley 2004) followed by clonal selection by fluorescence-activated cell sorting (FACS). Cells were maintained in Dulbecco's Modified Eagle Media (DMEM) supplemented with 10% fetal bovine serum (FBS).

HEK-CCR5 cells were seeded (20 000 cells/well) overnight in 384-well plates that had been pretreated with 10 μg/ml of polyornithine (37° C., 1 h). Cells were then loaded with Fluo4-AM (Invitrogen) according to the manufacturer's recommendations and incubated for 1 h at 37° C. Culture medium was removed and cells were washed with phosphate-buffered saline (PBS) and incubated in Assay buffer (143 mM NaCl, 6 mM KCl, 1 mM CaCl2, 1 mM MgCl2, 0.1% glucose, 20 mM HEPES, pH 7.4). Ca2+-dependent fluorescence measurements were carried out on a FDSS 384-well plate reader (Hammamatsu). Molecules were screened (n=4) at a single concentration (300 nM) at which PSC-RANTES gives a maximal signal ((2)Gaertner 2008). Signaling activity was expressed as a percentage of the value obtained for 300 nM PSC-RANTES reference standard (maximum signaling), after subtraction of the value obtained for 300 nM 5P12-RANTES reference standard (background signaling).

The panel of parallel-synthesized chemokine analogs was tested on a plate-based G protein signaling assay similar to that as previously described (Gaertner 2008), but with CCR5 expressed in a Human Embryonic Kidney (HEK) cell background instead of a HeLa cell background. Compounds were tested at a single Eₘₐₓ concentration (300 nM), and the signal obtained was expressed as a percentage of the signal obtained in the same experiment using reference standard samples of the CCR5 superagonist PSC-RANTES (100% signaling) and the non-signaling ligand 5P12-RANTES (0% signaling). Expressed on this scale (FIG. 7), compounds ranged in activity between −5% and 200%. Compounds were divided into three groups: absent or low signaling (0-25% signal), medium signaling (25-100% signal) and high signaling (over 100% signal). Divided in this way and compared with the Eₘₐₓ values obtained using corresponding reference standard chemokine analogs identified previously (Gaertner 2008), a good correlation was obtained (FIG. 8). This indicates that screening parallel-synthesized chemokine analogs produced by the method described in Example 4 for G protein signaling is sufficient to stratify parallel-synthesized chemokine analogs non-signaling, medium-signaling and highsignaling groups with reasonable accuracy. The medium-signaling group in this study contains a number of analogs considered as non-signaling molecules, and three compounds belonging to the group of high signaling molecules as previously described (Gaertner 2008). It has been noted that G protein-coupled receptor signaling responses to agonists can vary to some extent according to the cellular background used (Kenakin 2002), and this is the most likely explanation for the discrepancy between the results of this experiment and from the reference experiment.

### Example 7—Evaluating RANTES/CCL5 Analogs by Cell Surface Downmodulation Assay

The pharmacological activity of RANTES/CCL5 analogs as produced by the method described in Example 4 was determined by a cell surface antibody binding assay using a technique based on bystander bioluminescence resonance energy transfer (BRET) (Namkung 2016).

For this assay, CHO-CCR5-RLuc8/YFP-CAAX cells were used. These cells contain a CCR5 C-terminally tagged with a derivative of Renilla luciferase (Rluc8) coexpressed with YFP fused to the prenylation CAAX box of KRAS to direct plasma membrane expression (Namkung 2016). Proximity between CCR5-Rluc and cell surface YFP generates a BRET signal that is lost upon receptor internalization. To generate these cells, an open reading frame encoding CCR5 fused via its C-terminus to Renilla Luciferase 8 (RLuc8) was generated by PCR assembly and inserted into the pCDNA 3.1(−) expression vector using the XbaI and NotI sites. CHO-K1 cells were transfected with the pCDNA3.1(−)-CCR5-RLuc8 plasmid using X-tremeGENE™ HP DNA Transfection Reagent (Roche), and a clone of stably transfected CHO-CCR5-Rluc8 cells was isolated. An open reading frame encoding yellow fluorescent protein (YFP) appended with the prenylation CAAX box sequence (KKKKKKSKTKCVIM) from KRas (Namkung 2016) was inserted by Gibson Assembly® (New England Biolabs) into the FUGW lentiviral vector (Lois 2002) that had been digested at the BamHI and EcoRI sites to generate the FUGW-YFP-CAAX vector. CHO-CCR5-RLuc8 cells were transduced with FUGW-YFP-KRas, and a YFP-positive population was isolated by flow cytometry. The resulting CHO-CCR5-RLuc8/YFP-CAAX clone were maintained in Roswell Park Memorial Institute medium (RPMI) supplemented with 10% FBS and 1% Geneticin at 37° C., 5% CO2.

CHO-CCR5-RLuc8/YFP-CAAX cells were seeded overnight in 96 well-plates (20.000 cells/well), then medium was removed and replaced with chemokine analogs (300 nM) diluted in BRET Buffer (5 M NaCl, 1 M KCl, 100 mM MgSO4, 1 M HEPES, 20% Glucose, 1% bovine serum albumin, 5 pM Coelenterazine H). BRET measurements were performed on a Polarstar® (BMG Labtech) plate reader with a filter set (center wavelength/band width) of 475/30 nm (donor) and 535/30 nm (acceptor). Luminescence was recorded immediately after 25 min of incubation at 37° C., and BRET ratios, defined as emission from the acceptor YFP (535 nm) divided by from the donor RLuc8 (475 nm) were calculated. Molecules were screened (n=4) at a single Eₘₐₓ concentration (300 nM) (47). Internalization activity was expressed as a percentage of the value obtained for 300 nM PSC-RANTES reference standard (maximum internalization), after subtraction of the value obtained for 300 nM 5P12-RANTES reference standard (background internalization).

CHO-CCR5-RLuc8/YFP-CAAX cells were then used to measure the capacity of the RANTES/CCL5 parallel-synthesized chemokine analogs to elicit steady state downmodulation of CCR5. BRET signals in individual wells were recorded after 25 min incubation with parallel-synthesized chemokine analogs at a single Eₘₐₓ concentration (300 nM), and the level of receptor internalization was expressed as a percentage of the internalization signal obtained by reference standard samples of the CCR5 superagonist PSC-RANTES (100% internalization) and the non-internalizing ligand 5P12-RANTES (0% signaling). Expressed on this scale (FIG. 9), compounds ranged in activity between −10% and 115%. Compounds were divided into three groups: absent or low downmodulation (0-25%), medium downmodulation (25-80%) and high downmodulation (over 80%). Divided in this way and compared with the values obtained using corresponding reference standard chemokine analogs as previously described (Gaertner 2008), a good correlation was obtained (FIG. 10). This indicates that screening parallel-synthesized chemokine analogs produced by the method described in Example 4 for CCR5 downmodulation is suitable for rapidly and inexpensively stratifying parallel-synthesized chemokine analogs into non-signaling, medium-signaling and high-signaling groups.

### Example 8—Producing and Screening CCL25 Analogs

A series of 42 analogs of CCL25 were identified in a phage chemokine library selection experiment on cells expressing the cognate chemokine CCR9, using existing phage display techniques (Dorgham 2016; Hartley 2003). An additional 10 analogs of CCL25 were rationally designed (extension of the N-terminal region, alanine scanning mutagenesis). Thus, a group of 52 CCL25 analogs were identified. This group of 52 CCL25 analogs were then synthesized according the method of the present invention as described herein.

To provide the common, structurally invariant region of CCL25, a large batch of the C-terminal fragment of CCL25 corresponding to residues 8-74 of CCL25 was prepared by solid-phase peptide synthesis as described in Example 1. CCL25(8-74) constituted the Core Fragment, unchanged across the panel of analogs, that was used for downstream parallel ligation reactions with a plurality of N-terminal structurally variant regions of CCL25.

To provide a plurality of structurally variant regions of CCL25, the N-terminal regions of the wild-type CCL25 and of the 52 previously identified analogs were synthesized in parallel as described in Example 2. These Variant Region N-terminal peptides corresponded to residues1 to 7 of CCL25, and some of the variants include 1 or 2 additional amino acid extensions (Table 4).

To produce a plurality of complete, variant CCL25 analogs, the C-terminal Cys7 thioster residue on the Variant Region N-terminal peptides were ligated to the Cys8 N-terminal residue on the Core Fragment using the parallel in-well native chemical ligation reaction as described in Example 3. A sample of these analogs was assessed by HPLC (FIG. 11, 11A, 11B) and MS (Table 5) for purity and integrity. All but 2 of the 52 target analogs (1P27-CCL25 and 1P43-CCL25) were successfully synthesized. The CCL25 analogs were separated, folded, desalted, and lyophilized in parallel as described in Examples 3 and 4 to produce parallel samples each containing one of the CCL25 analogs.

Solutions were prepared from each sample the concentrations were normalized to an estimated concentration of 100 μM, based on the estimated purity and total protein concentration. These solutions were then used to prepare multi-well assay plates with each well containing a target CCL25 analog. Each target analog was screened in parallel at a single concentration (300 nM) for its ability to recruit arrestin-3 to CCR9 using a multi-well bioluminescence resonance energy transfer (BRET) assay on live cells, as shown in FIG. 12. The activity of the analogs was compared to reference standard 1 (Std1), a commercially sourced, purified recombinant CCL25 (1-127); reference standard 2 (Std2), a synthesized and purified CCL25 (1-74); and reference standards 2 and 3 (Std2 and Std3), CCL25 molecules (1-74) prepared using the method of the present invention. As expected, the purified CCL25 standard samples (Std1 and Std2) gave robust signals. Notably, the CCL25 standard samples produced comparable signals to the highly purified standard samples. Of the analogs (A1-F7) many had no detectable signaling activity, while some showed intermediate levels or levels higher than those of the parent compound.

These results indicate that the plurality of folded structurally variant polypeptides produced by the method of the present invention exhibit biological activity that can be detected in a screening assay. Furthermore, the biological activity of the folded structurally variant polypeptides produced by the method of the present invention are capable of exhibiting comparable or greater biological activity in an assay compared to more highly purified polypeptides produced by previously known methods.

### REFERENCES

Boto, A.; Gallardo, J. A.; Hernandez, D.; Hernandez, R. (2007) Synthesis of Unnatural Amino Acids from Serine Derivatives by β-Fragmentation of Primary Alkoxyl Radicals, Journal of Organic Chemistry 72(19), 7260-7269.

Canne, L.; Stephen, K. B. H.; Reyna, R. U.S. Pat. No. 7,094,871. Solid phase native chemical ligation of unprotected or N-terminal cysteine protected peptides in aqueous solution.

Cerini, F.; Landay, A.; Gichinga, C.; Lederman, M. M.; Flyckt, R.; Starks, D.; Offord, R. E.; Le Gal, F.; Hartley, O. (2008) Chemokine analogues show suitable stability for development as microbicides, Journal of Acquired Immune Deficiency Syndromes 49(5), 472-476.

Dawson, P. E.; Muir, T. W.; Clark-Lewis, I.; Kent, S. B. (1994) Synthesis of proteins by native chemical ligation, Science 266(5186), 776-779.

Dorgham, K.; Cerini, F.; Gaertner, H.; Melotti, A.; Rossitto-Boriat, I.; Gorochov, G.; Hartley, O. (2016) Generating Chemokine Analogs with Enhanced Pharmacological Properties Using Phage Display, Methods in Enzymology 570, 47-72.

Engelhard, M. (2016) Quest for the chemical synthesis of proteins, Journal of Peptide Science 22(5), 246-251.

Escola, J. M.; Kuenzi, G.; Gaertner, H.; Foti, M.; Hartley, O. (2010) CC chemokine receptor 5 (CCRS) desensitization: cycling receptors accumulate in the trans-Golgi network, Journal of Biological Chemistry 285, 41772-41780.

Gaertner, H.; Cerini, F.; Escola, J. M.; Kuenzi, G.; Melotti, A.; Offord, R.; Rossitto-Borlat, I.; Nedellec, R.; Salkowitz, J.; Gorochov, G.; Mosier, D.; Hartley, O. (2008) Highly potent, fully recombinant anti-HIV chemokines: reengineering a low-cost microbicide, Proceedings of the National Academy of Sciences of the United States of America 105(46), 17706-17711.

(2) Gaertner, H.; Lebeau, O.; Borlat, I.; Cerini, F.; Dufour, B.; Kuenzi, G.; Melotti, A.; Fish, R. J.; Offord, R.; Springael, J. Y.; Parmentier, M.; Hartley, O. (2008) Highly potent HIV inhibition: engineering a key anti-HIV structure from PSC-RANTES into MIP-1 beta/CCL4, Protein Engineering Design & Selection 21(2), 65-72.

Hartley, O.; Dorgham, K.; Perez-Bercoff, D.; Cerini, F.; Heimann, A.; Gaertner, H.; Offord, R. E.; Pancino, G.; Debre, P.; Gorochov, G. (2003) Human immunodeficiency virus type 1 entry inhibitors selected on living cells from a library of phage chemokines, Journal of Virology 77(12), 6637-6644.

Hartley, O.; Gaertner, H.; Wilken, J.; Thompson, D.; Fish, R.; Ramos, A.; Pastore, C.; Dufour, B.; Cerini, F.; Melotti, A.; Heveker, N.; Picard, L.; Alizon, M.; Mosier, D.; Kent, S.; Offord, R. (2004) Medicinal chemistry applied to a synthetic protein: development of highly potent HIV entry inhibitors, Proceedings of the National Academy of Sciences of the United States of America 101(47), 16460-16465.

He J.; Li S.; Deng Y.; Fu H.; Lafortez B. N.; Spangler J. E.; Homs A.; Yu J. Q. (2014) Ligand-Controlled C(sp3)-H Arylation and Olefination in Synthesis of Unnatural Chiral α-Amino Acids, Science 343(6176), 1216-1220.

Jaradat, D. M. M. (2018) Thirteen decades of peptide synthesis: key developments in solid phase peptide synthesis and amide bond formation utilized in peptide ligation, Amino Acids 50(1), 39-68.

Kenakin, T. (2002) Efficacy at G-protein-coupled receptors, Nature Reviews Drug Discovery 1(2), 103-110.

Kolb, H. C.; Finn, M. G.; Sharpless, K. B. (2001) Click Chemistry: Diverse Chemical Function from a Few Good Reactions, Angewandte Chemie (International ed. In English) 40(11), 2004-2021.

Snyder, L. R. (2000) HPLC: Past and Present, Analytical Chemistry, 72(11), 412-420.

Loeffler, F. F.; Foertsch, T. C.; Popov, R.; Mettes, D. S.; Schlageter M.; Sedlmayr, M.; Ridder, B.; Dang, F. X; von Bojnicic-Kninski, C.; Weber, L. K.; Fischer, A.; Greifenstein, J.; Bykovska, V.; Buliev, I.; Bischoff, F. R.; Hahn, L.; Meier, M. A. R., Brase, S.; Powell, A. K.; Balaban, T. S.; Breitling, F.; Nesterov-Mueller, A. (2016) High-Flexibility Combinatorial Peptide Synthesis with Laser-Based Transfer of Monomers in Solid Matrix Material, Nature Communications 7, 11844.

Loibl, S. F.; Harpaz, Z.; Zitterbart, R.; Seitz, O. (2016) Total chemical synthesis of proteins without HPLC purification, Chemical Science 7(11), 6753-6759.

Lois, C.; Hong, E. J.; Pease, S.; Brown, E. J.; Baltimore, D. (2002) Germline transmission and tissue-specific expression of transgenes delivered by lentiviral vectors, Science 295(5556), 868-872.

Low D.; Hu Y.; Botti P. WO2004105685. Displaceable Linker Solid Phase Chemical Ligation.

Merrifield, B. (1997) Concept and early development of solid-phase peptide synthesis, Methods Enzymol, 289, 3-13.

Mijalis, A. J.; Thomas, D. A. 3^{rd}; Simon, M. D.; Adamo, A.; Beamont, R.; Jensen, K. F.; Pentelute, B. L. (2017) A fully automated flow-based approach for accelerated peptide synthesis, Nature Chemical Biology, 13, 464-466.

Namkung, Y.; Le Gouill, C.; Lukashova, V.; Kobayashi, H.; Hogue, M.; Khoury, E.; Song, M.; Bouvier, M.; Laporte, S. A. (2016) Monitoring G protein-coupled receptor and beta-arrestin trafficking in live cells using enhanced bystander BRET, Nature Communications 7, 12178.

Ollivier, N.; Dheur, J.; Mhidia, R.; Blanpain, A.; Melnyk, O. (2010) Bis(2sulfanylethyl)amino Native Peptide Ligation, Organic Letters 12(22), 5238-5241.

Pleskoff, O.; Treboute, C.; Brelot, A.; Heveker, N.; Seman, M.; Alizon, M. (1997) Identification of a chemokine receptor encoded by human cytomegalovirus as a cofactor for HIV- 1 entry, Science 276(5320), 1874-1878.

Raibaut L.; El Mandi O.; Melnyk O. (2014) Solid Phase Protein Chemical Synthesis. In: Liu L. (eds) Protein Ligation and Total Synthesis II. Topics in Current Chemistry, 363, 103-154. Springer.

Roberge, J. Y.; Beebe, X.; Danishefsky, S. J. (1995) A strategy for a convergent synthesis of N-linked glycopeptides on a solid support, Science 269(5221), 202-204.

Simmons, G.; Clapham, P. R.; Picard, L.; Offord, R. E.; Rosenkilde, M. M.; Schwartz, T. W.; Buser, R.; Wells, T. N. C.; Proudfoot, A. E. (1997) Potent inhibition of HIV-1 infectivity in macrophages and lymphocytes by a novel CCRS antagonist, Science 276(5310), 276-279.

Tam, J. P.; Xu, J.; Eom, K. D. (2001) Methods and strategies of peptide ligation, Biopolymers 60(3), 194-205.

Zou, H.; Li, L.; Zhang, T.; Shi, M.; Zhang, N.; Huang. J.; Xian, M. (2018) Biosynthesis and biotechnological application of non-canonical amino acids: Complex and unclear, Biotechnology Advances 36(7), 1917-1927.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

The phrase “and/or,” as used herein in the specification and in the claims, should be understood to mean “either or both” of the elements so conjoined, i.e., elements that are conjunctively present in some cases and disjunctively present in other cases.

Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e. , the inclusion of at least one, but also including more than one, of a number or list of elements, and, optionally, additional unlisted items.

As used herein, whether in the specification or the appended claims, the transitional terms “comprising”, “including”, “carrying”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the invention disclosed and/or claimed herein.

TABLE 1

| | | | M_{calc} | M_{obs} | Difference | |
|---|---|---|---|---|---|---|
| Fragment | SEQ ID NO | Sequence | (Da) | (Da) | (Da) | Interpretation |
| Met | SEQ ID NO: 1 | MSPYSSDTTPC | 1306.6 | 1033.4 | −273.1 | Ac-Tyr4 truncation |
| 1P1 | SEQ ID NO: 2 | LSPVSSQSSAC | 1183.6 | 928.4 | −255.3 | Ac-Val4 truncation |
| 1P2 | SEQ ID NO: 3 | FSPLSSQSSAC | 1231.6 | 964.3 | −267.3 | Ac-Leu4 truncation |
| 1P4 | SEQ ID NO: 4 | WSPLSSQSPAC | 1280.6 | 952.4 | −328.3 | Ac-Leu4 truncation |
| 1P5 | SEQ ID NO: 5 | LSPQSSLSSSC | 1213.6 | 958.3 | −255.3 | Ac-Gln4 truncation |
| 1P6 | SEQ ID NO: 6 | ZSPGSSWSAAC | 1181.5 | 1020 | −161.5 | Ac-Pro3 truncation |
| 1P7 | SEQ ID NO: 7 | MSPLSSQASAC | 1199.6 | 1199.4 | −0.1 | |
| 2P1 | SEQ ID NO: 8 | FVPQSGQSTPC | 1268.6 | 1268.5 | −0.1 | |
| 2P2 | SEQ ID NO: 9 | LVPQPGQSTPC | 1244.7 | 1244.5 | −0.2 | |
| 2P3 | SEQ ID NO: 10 | ZGPPLMQTTPC | 1273.6 | 861.2 | −412.4 | Ac-Met6 truncation |
| | | | | | | +Na⁺ adduct |
| 2P4 | SEQ ID NO: 11 | MVPQSGQSTPC | 1252.6 | 1274.4 | 21.8 | +Na⁺ adduct |
| 2P5 | SEQ ID NO: 12 | ZGPPMMQTTPC | 1291.6 | 971.2 | −320.4 | Ac-Met5 truncation |
| 2P6 | SEQ ID NO: 13 | ZGPPGGQTTPC | 1143.6 | 1143.3 | −0.2 | |
| 2P7 | SEQ ID NO: 14 | FAPMSQQSTSC | 1304.6 | 1304.3 | −0.3 | |
| 2P8 | SEQ ID NO: 15 | ZGPLSGQSTPC | 1175.6 | 1175.4 | −0.2 | |
| 2P9 | SEQ ID NO: 16 | ZGPPGGQSTPC | 1129.5 | 1129.3 | −0.2 | |
| 2P10 | SEQ ID NO: 17 | ZGPPMMQSTPC | 1277.6 | 1277.3 | −0.3 | |
| 2P11 | SEQ ID NO: 18 | TGPPGGQSTPC | 1119.5 | 1119.3 | −0.2 | |
| 2P12 | SEQ ID NO: 19 | VGPLSQQATPC | 1218.7 | 1218.4 | −0.3 | |
| 2P13 | SEQ ID NO: 20 | ZFPPGGQSTPC | 1219.6 | 1219.3 | −0.3 | |
| 2P14 | SEQ ID NO: 21 | FAPMSQQSTPC | 1314.6 | 1314.6 | 0 | |
| 2P15 | SEQ ID NO: 22 | AAPLSQQSTPC | 1220.6 | 1220.4 | −0.2 | |
| 5P1 | SEQ ID NO: 23 | ZGPPLMWLQVC | 1372.7 | 960.4 | −412.3 | Ac-Met6 truncation |
| 5P2 | SEQ ID NO: 24 | ZGPPLMWLQSC | 1360.7 | 1382.3 | 21.6 | +Na⁺ adduct |
| 5P3 | SEQ ID NO: 25 | ZGPPLMWMQVC | 1390.7 | 1390.4 | −0.3 | |
| 5P4 | SEQ ID NO: 26 | ZGPPLMWMQSC | 1378.6 | 1378.6 | 0 | |
| 5P5 | SEQ ID NO: 27 | ZGPPLMVVTQVC | 1360.7 | 1360.4 | −0.3 | |
| 5P6 | SEQ ID NO: 28 | ZGPPLMVVTQSC | 1348.6 | 1348.4 | −0.3 | |
| 5P7 | SEQ ID NO: 29 | ZGPPLMALQSC | 1245.6 | 1245.4 | −0.2 | |
| 5P8 | SEQ ID NO: 30 | ZGPPLMSTQSC | 1249.6 | 1249.4 | −0.2 | |
| 5P9 | SEQ ID NO: 31 | ZGPPLMSFQSC | 1295.6 | 1295.4 | −0.2 | |
| 5P10 | SEQ ID NO: 32 | ZGPPLMWLQTC | 1374.7 | 1374.5 | −0.2 | |
| 5P11 | SEQ ID NO: 33 | ZGPPLMWRGSC | 1332.7 | 1332.4 | −0.3 | |
| 5P12 | SEQ ID NO: 34 | ZGPPLMATQSC | 1233.6 | 1234.5 | 0.9 | |
| 5P13 | SEQ ID NO: 35 | ZGPPLMWLGGC | 1259.6 | 1281.3 | 21.7 | +Na⁺ adduct |
| 5P14 | SEQ ID NO: 36 | ZGPPLMSLQVC | 1273.7 | 1274.6 | 0.9 | |
| 5P15 | SEQ ID NO: 37 | ZGPPLMSLSVC | 1232.6 | 1232.4 | −0.3 | |
| 5P16 | SEQ ID NO: 38 | ZGPPLMGLSVC | 1202.6 | 1202.4 | −0.2 | |
| 6P1 | SEQ ID NO: 39 | ZGPPGGGGLGC | 1000.5 | 1022.2 | 21.7 | +Na⁺ adduct |
| 6P2 | SEQ ID NO: 40 | ZGPPGDGGQVC | 1115.5 | 1115.3 | −0.2 | |
| 6P3 | SEQ ID NO: 41 | ZGPPGDGGSVC | 1074.5 | 1075.5 | 1 | |
| 6P4 | SEQ ID NO: 42 | ZGPPGDIVLAC | 1170.6 | 1171.5 | 0.9 | |
| 6P5 | SEQ ID NO: 43 | ZGPPGGGGQSC | 1045.5 | 1045.3 | −0.2 | |
| 6P6 | SEQ ID NO: 44 | ZGPPGGGGTRC | 1087.5 | 1087.3 | −0.2 | |
| 6P7 | SEQ ID NO: 45 | ZGPPGSWSSVC | 1205.6 | 1205.3 | −0.2 | |
| 6P8 | SEQ ID NO: 46 | ZGPPMGGQVTC | 1175.6 | 1175.3 | −0.2 | |
| 6P9 | SEQ ID NO: 47 | ZGPPGDTYQAC | 1237.6 | 1237.4 | −0.2 | |
| 6P10 | SEQ ID NO: 48 | ZGPPGDTVLWC | 1273.6 | 1273.4 | −0.2 | |
| 6P11 | SEQ ID NO: 49 | ZGPPGSYDYSC | 1274.5 | 1274.3 | −0.3 | |
| 6P12 | SEQ ID NO: 50 | ZGPPLGAGSSC | 1074.5 | 1074.3 | −0.2 | |
| 6P13 | SEQ ID NO: 51 | ZGPPLGSMGPC | 1144.6 | 1144.3 | −0.3 | |
| 6P14 | SEQ ID NO: 52 | ZGPPLDFGGAC | 1162.6 | 1162.3 | −0.2 | |
| 6P15 | SEQ ID NO: 53 | ZGPPMGGTSAC | 1106.5 | 1107.4 | 0.9 | |
| 6P16 | SEQ ID NO: 54 | ZGPPMQGGLSC | 1175.6 | 1175.3 | −0.3 | |
| 6P17 | SEQ ID NO: 55 | ZGPPMMAGLSC | 1192.6 | 1192.3 | −0.3 | |
| 6P18 | SEQ ID NO: 56 | ZGPPLQASVTC | 1201.6 | 1201.4 | −0.3 | |
| 6P19 | SEQ ID NO: 57 | ZGPPMSGHSTC | 1202.5 | 1202.3 | −0.3 | |
| 6P20 | SEQ ID NO: 58 | ZGPPMSAYQVC | 1281.6 | 1281.3 | −0.3 | |
| 7P1 | SEQ ID NO: 59 | ZGPPGQWYQSC | 1351.6 | 1352.5 | 0.9 | |
| 7P2 | SEQ ID NO: 60 | ZGPPLSWSQVC | 1302.7 | 1302.4 | −0.3 | |
| 7P3 | SEQ ID NO: 61 | ZGPPGDWSQVC | 1274.6 | 1274.3 | −0.3 | |
| 7P6 | SEQ ID NO: 62 | ZGPPQGWSQVC | 1287.6 | 1287.4 | −0.3 | |
| 7P7 | SEQ ID NO: 63 | ZGPPQSWSQAC | 1289.6 | 1289.4 | −0.3 | |
| 7P8 | SEQ ID NO: 64 | ZGPPGQWGQVC | 1257.6 | 1257.4 | −0.3 | |
| 7P9 | SEQ ID NO: 65 | ZGPPGMWSQSC | 1278.6 | 1278.3 | −0.3 | |
| 7P11 | SEQ ID NO: 66 | ZGPPLQWMQVC | 1387.7 | 1387.5 | −0.2 | |
| 7P12 | SEQ ID NO: 67 | ZGPPLMWSQVC | 1346.7 | 1346.5 | −0.2 | |
| 7P13 | SEQ ID NO: 68 | ZGPPGQWSQVC | 1287.6 | 1287.4 | −0.3 | |
| 7P14 | SEQ ID NO: 69 | ZGPPLQWMQAC | 1359.7 | 1359.4 | −0.3 | |
| 7P15 | SEQ ID NO: 70 | ZGPPLQWFQVC | 1403.7 | 1403.5 | −0.3 | |
| 7P16 | SEQ ID NO: 71 | ZGPPLQVVTQVC | 1357.7 | 1357.5 | −0.2 | |
| 7P19 | SEQ ID NO: 72 | ZGPPLSWLQVC | 1328.7 | 1328.5 | −0.2 | |
| 8P2 | SEQ ID NO: 73 | ZGPLSQASQVC | 1218.6 | 1218.3 | −0.3 | |
| 8P3 | SEQ ID NO: 74 | ZGPLSQAFQVC | 1278.7 | 1278.4 | −0.3 | |
| 8P4 | SEQ ID NO: 75 | ZGPLSQSSQVC | 1234.6 | 1234.4 | −0.3 | |
| 8P5 | SEQ ID NO: 76 | ZGPLSSQSQVC | 1234.6 | 1234.4 | −0.3 | |
| 8P6 | SEQ ID NO: 77 | ZGPLSGWAQVC | 1246.6 | 1246.4 | −0.3 | |
| 8P8 | SEQ ID NO: 78 | ZGPLSQWQQVC | 1374.7 | 1374.4 | −0.3 | |
| M7 | SEQ ID NO: 79 | ZGPYSSDTTPC | 1256.6 | 1278.3 | 21.7 | +Na⁺ adduct |
| M9 | SEQ ID NO: 80 | MSPPLSDTTPC | 1266.6 | 1266.3 | −0.3 | |
| M10 | SEQ ID NO: 81 | MSPYSMQTTPC | 1363.6 | 1363.8 | 0.2 | |
| M12 | SEQ ID NO: 82 | MSPLSSWLQVC | 1368.7 | 1390.4 | 21.7 | +Na⁺ adduct |
| M13 | SEQ ID NO: 83 | MSPLSSQAQVC | 1268.6 | 1290.3 | 21.7 | +Na⁺ adduct |
| M15 | SEQ ID NO: 84 | ZGPLSGWLQVC | 1288.7 | 1288.4 | −0.3 | |
| M19 | SEQ ID NO: 85 | ZGPLSGQSQVC | 1204.6 | 1204.3 | −0.3 | |
| M20 | SEQ ID NO: 86 | ZGPPGDWLQVC | 1300.6 | 1300.4 | −0.3 | |
| M21 | SEQ ID NO: 87 | ZGPPLMSVLAC | 1216.7 | 1216.3 | −0.3 | |
| M22 | SEQ ID NO: 88 | ZGPPLMGLQVC | 1243.7 | 1243.4 | −0.3 | |
| M23 | SEQ ID NO: 89 | ZGPPLMALQVC | 1257.7 | 1279.4 | 21.7 | +Na⁺ adduct |
| M27 | SEQ ID NO: 90 | ZGPPLMRLQVC | 1342.7 | 1343.6 | 0.9 | |
| M28 | SEQ ID NO: 91 | ZGPPLMTLQVC | 1287.7 | 1287.4 | −0.3 | |
| M36 | SEQ ID NO: 92 | ZGPPLMVTQSC | 1261.6 | 1261.3 | −0.3 | |
| M37 | SEQ ID NO: 93 | ZGPPLMSLQSC | 1261.6 | 1261.3 | −0.3 | |
| M39 | SEQ ID NO: 94 | ZGPPLMSGQSC | 1205.6 | 1205.3 | −0.3 | |
| M40 | SEQ ID NO: 95 | ZGPPLMSSQSC | 1235.6 | 1235.4 | −0.2 | |
| M44 | SEQ ID NO: 96 | ZGPPLMSLTVC | 1246.7 | 1246.4 | −0.3 | |

TABLE 2A

| Target | M_{calc} (Da) | M_{obs} (Da) | Differemce (Da) |
|---|---|---|---|
| 2P1-RANTES | 7 [symbol] 41 | 7952 | +11 |
| 2P2-RANTES | 7 [symbol] 17 | 7927 | +10 |
| 2P7-RANTES | 7 [symbol] 77 | 7967 | +10 |
| 2P10-RANTES | 7 [symbol] 50 | 7960 | +10 |
| 2P12-RANTES | 7 [symbol] 91 | 7900 | + [symbol] |
| 2P13-RANTES | 7 [symbol] 92 | 7901 | + [symbol] |
| 2P15-RANTES | 7 [symbol] 93 | 7903 | +10 |
| 5P2-RANTES | 8033 | 8045 | +12 |
| 5P3-RANTES | 8063 | 8078 | +15 |
| 5P4-RANTES | 8051 | 8061 | +10 |
| 5P5-RANTES | 8033 | 8042 | + [symbol] |
| 5P7-RANTES | 7 [symbol] 18 | 7927 | + [symbol] |
| 5P8-RANTES | 7 [symbol] 22 | 7931 | + [symbol] |
| 5P9-RANTES | 7 [symbol] 68 | 7978 | +10 |
| 5P10-RANTES | 8047 | 8057 | +10 |
| 5P14-RANTES | 7 [symbol] 46 | 795 [symbol] | +10 |
| 5P15-RANTES | 7 [symbol] 05 | 7915 | +10 |
| 6P6-RANTES | 7760 | 7768 | +8 |
| 6P9-RANTES | 7910 | 7919 | +9 |
| 6P11-RANTES | 7 [symbol] 47 | 7955 | +8 |
| 6P12-RANTES | 7747 | 7755 | +8 |
| 6P13-RANTES | 7 [symbol] 17 | 7824 | +7 |
| 6P14-RANTES | 7 [symbol] 35 | 7 [symbol] 43 | +8 |
| 6P16-RANTES | 7 [symbol] 48 | 7857 | +9 |
| 6P17-RANTES | 7 [symbol] 65 | 7874 | +9 |
| 6P18-RANTES | 7 [symbol] 74 | 7883 | +9 |
| 6P20-RANTES | 7 [symbol] 54 | 7964 | +10 |
| 7P3-RANTES | 7 [symbol] 47 | 7955 | +8 |
| 7P8-RANTES | 7 [symbol] 30 | 7938 | +8 |
| 7P9-RANTES | 7 [symbol] 51 | 7960 | +9 |
| 7P11-RANTES | 8060 | 8070 | +10 |
| 7P12-RANTES | 801 [symbol] | 8030 | +11 |
| 7P13-RANTES | 7 [symbol] 60 | 7971 | +11 |
| 7P15-RANTES | 8076 | 8086 | +10 |
| 7P16-RANTES | 8030 | 8040 | +10 |
| 8P3-RANTES | 7 [symbol] 51 | 7960 | +9 |
| 8P4-RANTES | 7907 | 7916 | +9 |
| M10-RANTES | 8036 | 8051 | +15 |
| M12-RANTES | 8041 | 8050 | +9 |
| M13-RANTES | 7 [symbol] 41 | 7956 | +15 |
| M20-RANTES | 7 [symbol] 93 | 7983 | +10 |
| M21-RANTES | 7 [symbol] 89 | 7905 | +16 |
| M22-RANTES | 7 [symbol] 16 | 7926 | +10 |
| M23-RANTES | 7930 | 7940 | +10 |
| M28-RANTES | 7960 | 7968 | +8 |
| M36-RANTES | 7934 | 7948 | +14 |
| M37-RANTES | 7934 | 7948 | +14 |
| M38-RANTES | 7878 | 7887 | +9 |
| M40-RANTES | 7908 | 7918 | +10 |

[symbol]

indicates data missing or illegible when filed

TABLE 2B

| | | M_{obs}1 (Da) | M_{obs}2 (Da) |
|---|---|---|---|
| Target | M_{calc} (Da) | (Difference) | (Difference) |
| 1P7-RANTES | 7872 | 7 [symbol] [symbol] 4 (+12) | 7896 (+26) |
| 2P4-RANTES | 7925 | 7939 (+14) | 7945 (+20) |
| 2P6-RANTES | 7816 | 7826 (+10) | 7 [symbol] 37 (+21) |
| 2P8-RANTES | 7848 | 785 [symbol] (+10) | 7 [symbol] [symbol] 7 (+19) |
| 2P9-RANTES | 7802 | 7 [symbol] 11 (+9) | 7 [symbol] 20 (+1 [symbol] ) |
| 2P11-RANTES | 7792 | 7804 (+12) | 7818 (+26) |
| 2P14-RANTES | 7987 | 7 [symbol] [symbol] 6 (+ [symbol] ) | 8005 (+18) |
| 5P6-RANTES | 8021 | 8030 (+ [symbol] ) | 8047 (+26) |
| 5P11-RANTES | 8005 | 8012 (+7) | 8030 (+25) |
| 5P12-RANTES | 7 [symbol] 06 | 7921 (+15) | 7931 (+25) |
| 5P13-RANTES | 7 [symbol] 32 | 7942 (+10) | 7948 (+16) |
| 5P16-RANTES | 7 [symbol] 75 | 7883 (+ [symbol] ) | 7899 (+24) |
| 6P1-RANTES | 7672 | 7685 (+13) | 7696 (+26) |
| 6P2-RANTES | 7788 | 7805 (+17) | 7814 (+26) |
| 6P3-RANTES | 7746 | 775 [symbol] (+13) | 7775 (+29) |
| 6P4-RANTES | 7 [symbol] 43 | 7853 (+10) | 7861 (+18) |
| 6P5-RANTES | 7717 | 7727 (+10) | 7735 (+1 [symbol] ) |
| 6P7-RANTES | 7878 | 7890 (+12) | 7903 (+25) |
| 6P8-RANTES | 7848 | 7861 (+13) | 7873 (+25) |
| 6P10-RANTES | 7946 | 7955 (+9) | 7966 (+20) |
| 6P15-RANTES | 7778 | 7794 (+16) | 7 [symbol] 06 (+2 [symbol] ) |
| 6P19-RANTES | 7875 | 7886 (+11) | 7894 (+19) |
| 7P1-RANTES | 8024 | 8041 (+17) | 8051 (+27) |
| 7P2-RANTES | 7975 | 7 [symbol] [symbol] 1 (+16) | 8001 (+26) |
| 7P6-RANTES | 7960 | None | 7 [symbol] [symbol] [symbol] (+26) |
| 7P7-RANTES | 7962 | 7974 (+12) | 79 [symbol] 2 (+20) |
| 7P1 [symbol] -RANTES | 8001 | 8012 (+11) | 8022 (+21) |
| 8P2-RANTES | 7891 | 7900 (+9) | 7916 (+25) |
| 8P5-RANTES | 7907 | 7924 (+17) | 7 [symbol] 32 (+25) |
| 8P6-RANTES | 7919 | None | 7944 (+25) |
| 8P8-RANTES | 8047 | None | 8072 (+25) |
| M9-RANTES | 7939 | 7949 (+10) | 7 [symbol] [symbol] [symbol] (+19) |
| M15-RANTES | 7961 | 7978 (+17) | 7 [symbol] [symbol] 7 (+2 [symbol] ) |
| M19-RANTES | 7877 | 788 [symbol] (+9) | 7902 (+25) |
| M27-RANTES | 8015 | 8029 (+14) | 8041 (+26) |
| M44-RANTES | 7919 | 7928 (+9) | 7 [symbol] 3 [symbol] (+20) |

[symbol]

indicates data missing or illegible when filed

TABLE 3

| | | | | | Estimated | Estimated | Estimated | Estimated |
|---|---|---|---|---|---|---|---|---|
| | | % Peak | % Peak | | total | total | % yield | concentration |
| | Source of | area | area | Combined | protein | protein | of | in 250 μL |
| Target | Core | (target | (Met [symbol] | % target | concentration | content | target | solution |
| protein | Fragment | protein) | (O) variant) | peak area | (μM) | (nmol) | protein | (μM) |
| 5P7- | Core | 23.9 | — | 23.9 | 193 | 48 | 11.5 | 46 |
| RANTES | Fragment | | | | | | | |
| 6P9 | batch 1 | 19.5 | — | 19.5 | 239 | 60 | 11.6 | 47 |
| RANTES | | | | | | | | |
| 5P2- | | 17.4 | — | 17.4 | 149 | 37 | 6.5 | 26 |
| RANTES | | | | | | | | |
| 5P12- | Core | 17.0 | 18.4 | 35.4 | 105 | 26 | 9.3 | 37 |
| RANTES | Fragment | | | | | | | |
| 5P6- | batch 2 | 5.6 | 11.8 | 17.5 | 171 | 43 | 7.5 | 30 |
| RANTES | | | | | | | | |
| 7P1- | | 27.8 | 28.2 | 56.0 | 100 | 25 | 14.0 | 56 |
| RANTES | | | | | | | | |

[symbol]

indicates data missing or illegible when filed

TABLE 4

| | N-terminal |
|---|---|
| Target code | fragment sequence |
| | Position 1-2-3-4-5-6-7 |
| 1P1-CCL25 | (SEQ ID NO: 97) |
| | Z-G-A-L-R-Q-C |
| 1P2-CCL25 | (SEQ ID NO: 98) |
| | Z-G-V-A-R-N-C |
| 1P3-CCL25 | (SEQ ID NO: 99) |
| | Z-G-V-A-R-R-C |
| 1P4-CCL25 | (SEQ ID NO: 100) |
| | Z-G-V-Q-R-I-C |
| 1P6-CCL25 | (SEQ ID NO: 101) |
| | Y-Q-A-S-E-D-C |
| 1P7-CCL25 | (SEQ ID NO: 102) |
| | Y-Q-S-R-E-D-C |
| 1P8-CCL25 | (SEQ ID NO: 103) |
| | Y-S-Q-R-E-D-C |
| 1P9-CCL25 | (SEQ ID NO: 104) |
| | Z-G-A-F-Q-P-D-C |
| 1P10-CCL25 | (SEQ ID NO: 105) |
| | Z-G-G-F-K-Q-D-C |
| 1P12-CCL25 | (SEQ ID NO: 106) |
| | Z-G-F-L-T-A-D-C |
| 1P14-CCL25 | (SEQ ID NO: 107) |
| | Z-G-L-L-Q-Q-D-C |
| 1P16-CCL25 | (SEQ ID NO: 108) |
| | K-D-L-Q-F-E-D-C |
| 1P17-CCL25 | (SEQ ID NO: 109) |
| | L-D-A-Q-F-E-D-C |
| 1P18-CCL25 | (SEQ ID NO: 110) |
| | T-D-I-Q-F-E-D-C |
| 1P19-CCL25 | (SEQ ID NO: 111) |
| | V-D-G-Q-F-E-D-C |
| 1P21-CCL25 | (SEQ ID NO: 112) |
| | E-F-L-R-F-E-D-C |
| 1P22-CCL25 | (SEQ ID NO: 113) |
| | G-Q-L-K-F-E-D-C |
| 1P23-CCL25 | (SEQ ID NO: 114) |
| | I-T-Q-R-F-E-D-C |
| 1P24-CCL25 | (SEQ ID NO: 115) |
| | S-I-Q-R-F-E-D-C |
| 1P25-CCL25 | (SEQ ID NO: 116) |
| | Z-G-I-Q-F-I-D-C |
| 1P27-CCL25 | (SEQ ID NO: 117) |
| | Z-G-I-W-I-I-D-C |
| 1P28-CCL25 | (SEQ ID NO: 118) |
| | Z-G-I-W-Q-Y-D-C |
| 1P29-CCL25 | (SEQ ID NO: 119) |
| | Z-G-V-Q-Y-G-D-C |
| 1P30-CCL25 | (SEQ ID NO: 120) |
| | Z-L-L-W-F-E-D-C |
| 1P31-CCL25 | (SEQ ID NO: 121) |
| | Z-G-D-I-Q-P-D-C |
| 1P32-CCL25 | (SEQ ID NO: 122) |
| | Z-G-D-Q-P-I-D-C |
| 1P34-CCL25 | (SEQ ID NO: 123) |
| | R-R-A-E-E-D-C |
| 1P35-CCL25 | (SEQ ID NO: 124) |
| | R-R-K-Q-E-D-C |
| 1P36-CCL25 | (SEQ ID NO: 125) |
| | Z-G-K-S-Q-G-C |
| 1P37-CCL25 | (SEQ ID NO: 126) |
| | Z-G-R-Q-A-Q-C |
| 1P38-CCL25 | (SEQ ID NO: 127) |
| | Z-G-R-S-Q-Q-C |
| 1P39-CCL25 | (SEQ ID NO: 128) |
| | Z-S-K-R-E-D-C |
| 1P40-CCL25 | (SEQ ID NO: 129) |
| | Z-Y-K-Q-E-D-C |
| 1P41-CCL25 | (SEQ ID NO: 130) |
| | Z-G-A-W-W-R-C |
| 1P42-CCL25 | (SEQ ID NO: 131) |
| | Z-G-E-L-H-Q-C |
| 1P43-CCL25 | (SEQ ID NO: 132) |
| | Z-G-Q-V-W-L-C |
| 1P44-CCL25 | (SEQ ID NO: 133) |
| | Z-G-Q-W-S-G-C |
| 1P45-CCL25 | (SEQ ID NO: 134) |
| | Z-G-Q-Y-L-D-D-C |
| 1P46-CCL25 | (SEQ ID NO: 135) |
| | Z-G-S-Q-L-Q-D-C |
| 1P47-CCL25 | (SEQ ID NO: 136) |
| | G-R-D-Q-F-E-D-C |
| 1P48-CCL25 | (SEQ ID NO: 137) |
| | G-R-E-Q-F-E-D-C |
| 1P50-CCL25 | (SEQ ID NO: 138) |
| | V-Q-R-L-E-D-C |
| CCL25 | (SEQ ID NO: 139) |
| | Q-G-V-F-E-D-C |
| Z1A-CCL25 | (SEQ ID NO: 140) |
| | A-G-V-F-E-D-C |
| G2A-CCL25 | (SEQ ID NO: 141) |
| | Q-A-V-F-E-D-C |
| V3A-CCL25 | (SEQ ID NO: 142) |
| | Q-D-A-F-E-D-C |
| F4A-CCL25 | (SEQ ID NO: 143) |
| | Q-G-V-A-E-D-C |
| ESA-CCL25 | (SEQ ID NO: 144) |
| | Q-G-V-F-A-D-C |
| D6A-CCL25 | (SEQ ID NO: 145) |
| | Q-G-V-F-E-A-C |
| V3AV-CCL25 | (SEQ ID NO: 146) |
| | Q-G-A-V-F-E-D-C |
| E5AE-CCL25 | (SEQ ID NO: 147) |
| | Q-G-V-F-A-E-D-C |
| D6DA-CCL25 | (SEQ ID NO: 148) |
| | Q-G-V-F-E-D-A-C |
| D6DAS-CCL25 | (SEQ ID NO: 149) |
| | Q-G-V-F-E-D-A-S-C |

TABLE 5

| | | Expected | |
|---|---|---|---|
| | Expected | average MW | MW |
| Name | average MW | with Met⁶⁵(O) | observed |
| 1P1-CCL25 | 8436.95 | 8452.95 | 8445.96 |
| 1P2-CCL25 | 8408.89 | 8424.89 | 8407.17 |
| 1P3-CCL25 | 8450.98 | 8466.98 | 8456.84 |
| 1P4-CCL25 | 8465.00 | 8481.00 | 8480.93 |
| 1P6-CCL25 | 8493.88 | 8509.88 | 8498.47 |
| 1P7-CCL25 | 8578.99 | 8594.99 | 8571.21 |
| 1P8-CCL25 | 8578.99 | 8594.99 | 8586.7 |
| 1P9-CCL25 | 8526.98 | 8542.98 | 8539.66 |
| 1P10-CCL25 | 8544.01 | 8560.01 | 8553.69 |
| 1P12-CCL25 | 8516.00 | 8532.00 | 8528.48 |
| 1P14-CCL25 | 8566.06 | 8582.06 | 8572.32 |
| 1P16-CCL25 | 8676.15 | 8692.15 | 8689.66 |
| 1P17-CCL25 | 8619.06 | 8635.06 | 8626.28 |
| 1P18-CCL25 | 8649.08 | 8665.08 | 8664.28 |
| 1P19-CCL25 | 8591.00 | 8607.00 | 8596.12 |
| 1P21-CCL25 | 8737.24 | 8753.24 | 8741.42 |
| 1P22-CCL25 | 8618.12 | 8634.12 | 8624.35 |
| 1P23-CCL25 | 8690.18 | 8706.18 | 8697.36 |
| 1P24-CCL25 | 8676.16 | 8692.16 | 8688.96 |
| 1P25-CCL25 | 8585.11 | 8601.11 | 8578.67 |
| 1P27-CCL25 | 8609.17 | 8625.17 | not found |
| 1P28-CCL25 | 8674.15 | 8690.15 | 8694.18 |
| 1P29-CCL25 | 8530.96 | 8546.96 | 8546.3 |
| 1P30-CCL25 | 8715.25 | 8731.25 | 8732.64 |
| 1P31-CCL25 | 8536.97 | 8552.97 | 8539.5 |
| 1P32-CCL25 | 8536.97 | 8552.97 | 8547.86 |
| 1P34-CCL25 | 8556.99 | 8572.99 | 8564.11 |
| 1P35-CCL25 | 8613.10 | 8629.10 | 8613.21 |
| 1P36-CCL25 | 8368.82 | 8384.82 | 8380.68 |
| 1P37-CCL25 | 8451.92 | 8467.92 | 8457.79 |
| 1P38-CCL25 | 8467.92 | 8483.92 | 8482.92 |
| 1P39-CCL25 | 8526.98 | 8542.98 | 8533.82 |
| 1P40-CCL25 | 8575.02 | 8591.02 | 8583.57 |
| 1P41-CCL25 | 8568.08 | 8584.08 | 8588.52 |
| 1P42-CCL25 | 8475.94 | 8491.94 | 8489.86 |
| 1P43-CCL25 | 8495.03 | 8511.03 | not found |
| 1P44-CCL25 | 8426.86 | 8442.86 | 8432.04 |
| 1P45-CCL25 | 8603.03 | 8619.03 | 8611.04 |
| 1P46-CCL25 | 8539.98 | 8555.98 | 8543.35 |
| 1P47-CCL25 | 8648.06 | 8664.06 | 8642.2 |
| 1P48-CCL25 | 8662.09 | 8678.09 | 8665.55 |
| 1P50-CCL25 | 8541.03 | 8557.03 | 8550.91 |
| CCL25 | 8458.90 | 8474.90 | 8458.37 |
| Z1A-CCL25 | 8418.86 | 8434.86 | 8419.95 |
| G2A-CCL25 | 8472.93 | 8488.93 | 8473.12 |
| V3A-CCL25 | 8430.85 | 8446.85 | 8424.59 |
| F4A-CCL25 | 8382.81 | 8398.81 | 8376.18 |
| E5A-CCL25 | 8400.87 | 8416.87 | 8395.02 |
| D6A-CCL25 | 8414.89 | 8430.89 | 8400.18 |
| V3AV-CCL25 | 8529.98 | 8545.98 | 8528.15 |
| E5AE-CCL25 | 8529.98 | 8545.98 | 8535.03 |
| D6DA-CCL25 | 8529.98 | 8545.98 | 8528.61 |
| D6DAS-CCL25 | 8617.06 | 8633.06 | 8616.63 |

1. A method for producing a plurality of structurally variant polypeptide molecules in parallel comprising:
a. providing a plurality of structurally variant regions of a polypeptide molecule in parallel;
b. ligating each of said plurality of structurally variant regions of a polypeptide molecule to a common, structurally invariant region of said polypeptide molecule in parallel separate ligation reactions to produce a plurality of structurally variant polypeptide molecules; and
c. applying conditions to each of said separate ligation reactions in parallel to separate said plurality of structurally variant polypeptide molecules from each of said separate ligation reactions.

2. The method of claim 1, wherein after step ‘c’ the method further comprises:
d. folding each of said plurality of structurally variant polypeptide molecules in parallel separate folding reactions to produce a plurality of folded structurally variant polypeptide molecules; and
e. applying conditions to each of said separate folding reactions in parallel to separate said plurality of folded structurally variant polypeptide molecules from said folding reactions.

3. The method according to claim 1, wherein providing a plurality of structurally variant regions of a polypeptide molecule in parallel is performed column-free.

4. The method according to claim 1, wherein the conditions applied to each of said separate ligation reactions in parallel comprise column-free separation.

5. The method according to claim 2, wherein the folding comprises oxidative folding.

6. The method according to claim 2, wherein the conditions applied to each of said separate folding reactions in parallel comprise column-free separation.

7. The method according to claim 2, wherein the plurality of folded structurally variant polypeptide molecules is lyophilized in parallel after step ‘e’.

8. The method according to claim 1, wherein the plurality of structurally variant polypeptide molecules is lyophilized in parallel after step ‘c’.

9. The method according to claim 8, wherein the lyophilized polypeptide molecules are suspended with a solvent after lyophilization.

10. The method according to claim 1, wherein all parallel steps are column-free.

11. A method for determining at least one effect of each of a plurality of structurally variant polypeptide molecules in parallel comprising:
a. providing a plurality of structurally variant polypeptide molecules by the method defined in claim 1;
b. contacting the plurality of structurally variant polypeptide molecules separately in parallel with cells; and
c. determining at least one effect of each of the plurality of structurally variant polypeptide molecules on said cells.

12. The method according to claim 11, wherein the cells are selected from bacteria, genetically modified primary eukaryotic cells, transformed eukaryotic cells, and immortal eukaryotic cells.

13. The method according to claim 11, wherein the at least one effect is determined by a method selected from flow cytometry, polymerase chain reaction, real-time polymerase chain reaction, reverse-transcription polymerase chain reaction, western blot, enzyme-linked immunosorbent assay, enzyme-linked immunospot assay, cell migration assay, cell proliferation assay, cytotoxic killing assay, genome-wide sequencing, exome sequencing, RNA sequencing, chromatin immunoprecipitation sequencing, cell fusion assay, calcium flux assay, and cell surface antibody binding assay.

14. A method for determining at least one property of a plurality of folded structurally variant polypeptide molecules in parallel comprising:
a. providing a plurality of folded structurally variant polypeptide molecules by the method defined in claim 1;
b. determining at least one property of each of the plurality of folded structurally variant polypeptide molecules.

15. The method according to claim 14, wherein the at least one property is determined by a method selected from flow cytometry, polymerase chain reaction, real-time polymerase chain reaction, reverse-transcription polymerase chain reaction, western blot, enzyme-linked immunosorbent assay, enzyme-linked immunospot assay, cell migration assay, cell proliferation assay, cytotoxic killing assay, genome-wide sequencing, exome sequencing, RNA sequencing, chromatin immunoprecipitation sequencing, cell fusion assay, calcium flux assay, and cell surface antibody binding assay.

16. The method according to claim 1, wherein the plurality of structurally variant polypeptide molecules are proteins.

17. The method according to claim 16, wherein the plurality of structurally variant regions of said polypeptide molecule corresponds to a region of a protein, and wherein the common, structurally invariant region of said polypeptide molecule corresponds to a region of the same protein.

18. The method according to claim 16, wherein the plurality of structurally variant regions of said polypeptide molecule corresponds to a region of a first protein, and wherein the common, structurally invariant region of said polypeptide molecule corresponds to a region of a second protein.

19. The method according to claim 16, wherein the plurality of structurally variant regions of said polypeptide molecule are artificial polypeptides, and wherein the common, structurally invariant region of said polypeptide molecule corresponds to a region of a protein.

20. The method according to claim 16, wherein the plurality of structurally variant regions of said polypeptide molecule corresponds to a region of a protein, and wherein the common, structurally invariant region of said polypeptide molecule is an artificial polypeptide.


SEQUENCE LISTING (149 sequences)

SEQ ID NO: 1 - protein, 11 aa, Artificial Sequence
  synthetic
MSPYSSDTTPC

SEQ ID NO: 2 - protein, 11 aa, Artificial Sequence
  synthetic
LSPVSSQSSAC

SEQ ID NO: 3 - protein, 11 aa, Artificial Sequence
  synthetic
FSPLSSQSSAC

SEQ ID NO: 4 - protein, 11 aa, Artificial Sequence
  synthetic
WSPLSSQSPAC

SEQ ID NO: 5 - protein, 11 aa, Artificial Sequence
  synthetic
LSPQSSLSSSC

SEQ ID NO: 6 - protein, 11 aa, Artificial Sequence
  synthetic
  Xaa (1)..(1): Xaa is pyroglutamate
XSPGSSWSAAC

SEQ ID NO: 7 - protein, 11 aa, Artificial Sequence
  synthetic
MSPLSSQASAC

SEQ ID NO: 8 - protein, 11 aa, Artificial Sequence
  synthetic
FVPQSGQSTPC

SEQ ID NO: 9 - protein, 11 aa, Artificial Sequence
  synthetic
LVPQPGQSTPC

SEQ ID NO: 10 - protein, 11 aa, Artificial Sequence
  synthetic
  Xaa (1)..(1): Xaa is pyroglutamate
XGPPLMQTTPC

SEQ ID NO: 11 - protein, 11 aa, Artificial Sequence
  synthetic
MVPQSGQSTPC

SEQ ID NO: 12 - protein, 11 aa, Artificial Sequence
  synthetic
  Xaa (1)..(1): Xaa is pyroglutamate
XGPPMMQTTPC

SEQ ID NO: 13 - protein, 11 aa, Artificial Sequence
  synthetic
  Xaa (1)..(1): Xaa is pyroglutamate
XGPPGGQTTPC

SEQ ID NO: 14 - protein, 11 aa, Artificial Sequence
  synthetic
FAPMSQQSTSC

SEQ ID NO: 15 - protein, 11 aa, Artificial Sequence
  synthetic
  Xaa (1)..(1): Xaa is pyroglutamate
XGPLSGQSTPC

SEQ ID NO: 16 - protein, 11 aa, Artificial Sequence
  synthetic
  Xaa (1)..(1): Xaa is pyroglutamate
XGPPGGQSTPC

SEQ ID NO: 17 - protein, 11 aa, Artificial Sequence
  synthetic
  Xaa (1)..(1): Xaa is pyroglutamate
XGPPMMQSTPC

SEQ ID NO: 18 - protein, 11 aa, Artificial Sequence
  synthetic
TGPPGGQSTPC

SEQ ID NO: 19 - protein, 11 aa, Artificial Sequence
  synthetic
VGPLSQQATPC

SEQ ID NO: 20 - protein, 11 aa, Artificial Sequence
  synthetic
  Xaa (1)..(1): Xaa is pyroglutamate
XFPPGGQSTPC

SEQ ID NO: 21 - protein, 11 aa, Artificial Sequence
  synthetic
FAPMSQQSTPC

SEQ ID NO: 22 - protein, 11 aa, Artificial Sequence
  synthetic
AAPLSQQSTPC

SEQ ID NO: 23 - protein, 11 aa, Artificial Sequence
  synthetic
  Xaa (1)..(1): Xaa is pyroglutamate
XGPPLMWLQVC

SEQ ID NO: 24 - protein, 11 aa, Artificial Sequence
  synthetic
  Xaa (1)..(1): Xaa is pyroglutamate
XGPPLMWLQSC

SEQ ID NO: 25 - protein, 11 aa, Artificial Sequence
  synthetic
  Xaa (1)..(1): Xaa is pyroglutamate
XGPPLMWMQVC

SEQ ID NO: 26 - protein, 11 aa, Artificial Sequence
  synthetic
  Xaa (1)..(1): Xaa is pyroglutamate
XGPPLMWMQSC

SEQ ID NO: 27 - protein, 11 aa, Artificial Sequence
  synthetic
  Xaa (1)..(1): Xaa is pyroglutamate
XGPPLMWTQVC

SEQ ID NO: 28 - protein, 11 aa, Artificial Sequence
  synthetic
  Xaa (1)..(1): Xaa is pyroglutamate
XGPPLMWTQSC

SEQ ID NO: 29 - protein, 11 aa, Artificial Sequence
  synthetic
  Xaa (1)..(1): Xaa is pyroglutamate
XGPPLMALQSC

SEQ ID NO: 30 - protein, 11 aa, Artificial Sequence
  synthetic
  Xaa (1)..(1): Xaa is pyroglutamate
XGPPLMSTQSC

SEQ ID NO: 31 - protein, 11 aa, Artificial Sequence
  synthetic
  Xaa (1)..(1): Xaa is pyroglutamate
XGPPLMSFQSC

SEQ ID NO: 32 - protein, 11 aa, Artificial Sequence
  synthetic
  Xaa (1)..(1): Xaa is pyroglutamate
XGPPLMWLQTC

SEQ ID NO: 33 - protein, 11 aa, Artificial Sequence
  synthetic
  Xaa (1)..(1): Xaa is pyroglutamate
XGPPLMWRGSC

SEQ ID NO: 34 - protein, 11 aa, Artificial Sequence
  synthetic
  Xaa (1)..(1): Xaa is pyroglutamate
XGPPLMATQSC

SEQ ID NO: 35 - protein, 11 aa, Artificial Sequence
  synthetic
  Xaa (1)..(1): Xaa is pyroglutamate
XGPPLMWLGGC

SEQ ID NO: 36 - protein, 11 aa, Artificial Sequence
  synthetic
  Xaa (1)..(1): Xaa is pyroglutamate
XGPPLMSLQVC

SEQ ID NO: 37 - protein, 11 aa, Artificial Sequence
  synthetic
  Xaa (1)..(1): Xaa is pyroglutamate
XGPPLMSLSVC

SEQ ID NO: 38 - protein, 11 aa, Artificial Sequence
  synthetic
  Xaa (1)..(1): Xaa is pyroglutamate
XGPPLMGLSVC

SEQ ID NO: 39 - protein, 11 aa, Artificial Sequence
  synthetic
  Xaa (1)..(1): Xaa is pyroglutamate
XGPPGGGGLGC

SEQ ID NO: 40 - protein, 11 aa, Artificial Sequence
  synthetic
  Xaa (1)..(1): Xaa is pyroglutamate
XGPPGDGGQVC

SEQ ID NO: 41 - protein, 11 aa, Artificial Sequence
  synthetic
  Xaa (1)..(1): Xaa is pyroglutamate
XGPPGDGGSVC

SEQ ID NO: 42 - protein, 11 aa, Artificial Sequence
  synthetic
  Xaa (1)..(1): Xaa is pyroglutamate
XGPPGDIVLAC

SEQ ID NO: 43 - protein, 11 aa, Artificial Sequence
  synthetic
  Xaa (1)..(1): Xaa is pyroglutamate
XGPPGGGGQSC

SEQ ID NO: 44 - protein, 11 aa, Artificial Sequence
  synthetic
  Xaa (1)..(1): Xaa is pyroglutamate
XGPPGGGGTRC

SEQ ID NO: 45 - protein, 11 aa, Artificial Sequence
  synthetic
  Xaa (1)..(1): Xaa is pyroglutamate
XGPPGSWSSVC

SEQ ID NO: 46 - protein, 11 aa, Artificial Sequence
  synthetic
  Xaa (1)..(1): Xaa is pyroglutamate
XGPPMGGQVTC

SEQ ID NO: 47 - protein, 11 aa, Artificial Sequence
  synthetic
  Xaa (1)..(1): Xaa is pyroglutamate
XGPPGDTYQAC

SEQ ID NO: 48 - protein, 11 aa, Artificial Sequence
  synthetic
  Xaa (1)..(1): Xaa is pyroglutamate
XGPPGDTVLWC

SEQ ID NO: 49 - protein, 11 aa, Artificial Sequence
  synthetic
  Xaa (1)..(1): Xaa is pyroglutamate
XGPPGSYDYSC

SEQ ID NO: 50 - protein, 11 aa, Artificial Sequence
  synthetic
  Xaa (1)..(1): Xaa is pyroglutamate
XGPPLGAGSSC

SEQ ID NO: 51 - protein, 11 aa, Artificial Sequence
  synthetic
  Xaa (1)..(1): Xaa is pyroglutamate
XGPPLGSMGPC

SEQ ID NO: 52 - protein, 11 aa, Artificial Sequence
  synthetic
  Xaa (1)..(1): Xaa is pyroglutamate
XGPPLDFGGAC

SEQ ID NO: 53 - protein, 11 aa, Artificial Sequence
  synthetic
  Xaa (1)..(1): Xaa is pyroglutamate
XGPPMGGTSAC

SEQ ID NO: 54 - protein, 11 aa, Artificial Sequence
  synthetic
  Xaa (1)..(1): Xaa is pyroglutamate
XGPPMQGGLSC

SEQ ID NO: 55 - protein, 11 aa, Artificial Sequence
  synthetic
  Xaa (1)..(1): Xaa is pyroglutamate
XGPPMMAGLSC

SEQ ID NO: 56 - protein, 11 aa, Artificial Sequence
  synthetic
  Xaa (1)..(1): Xaa is pyroglutamate
XGPPLQASVTC

SEQ ID NO: 57 - protein, 11 aa, Artificial Sequence
  synthetic
  Xaa (1)..(1): Xaa is pyroglutamate
XGPPMSGHSTC

SEQ ID NO: 58 - protein, 11 aa, Artificial Sequence
  synthetic
  Xaa (1)..(1): Xaa is pyroglutamate
XGPPMSAYQVC

SEQ ID NO: 59 - protein, 11 aa, Artificial Sequence
  synthetic
  Xaa (1)..(1): Xaa is pyroglutamate
XGPPGQWYQSC

SEQ ID NO: 60 - protein, 11 aa, Artificial Sequence
  synthetic
  Xaa (1)..(1): Xaa is pyroglutamate
XGPPLSWSQVC

SEQ ID NO: 61 - protein, 11 aa, Artificial Sequence
  synthetic
  Xaa (1)..(1): Xaa is pyroglutamate
XGPPGDWSQVC

SEQ ID NO: 62 - protein, 11 aa, Artificial Sequence
  synthetic
  Xaa (1)..(1): Xaa is pyroglutamate
XGPPQGWSQVC

SEQ ID NO: 63 - protein, 11 aa, Artificial Sequence
  synthetic
  Xaa (1)..(1): Xaa is pyroglutamate
XGPPQSWSQAC

SEQ ID NO: 64 - protein, 11 aa, Artificial Sequence
  synthetic
  Xaa (1)..(1): Xaa is pyroglutamate
XGPPGQWGQVC

SEQ ID NO: 65 - protein, 11 aa, Artificial Sequence
  synthetic
  Xaa (1)..(1): Xaa is pyroglutamate
XGPPGMWSQSC

SEQ ID NO: 66 - protein, 11 aa, Artificial Sequence
  synthetic
  Xaa (1)..(1): Xaa is pyroglutamate
XGPPLQWMQVC

SEQ ID NO: 67 - protein, 11 aa, Artificial Sequence
  synthetic
  Xaa (1)..(1): Xaa is pyroglutamate
XGPPLMWSQVC

SEQ ID NO: 68 - protein, 11 aa, Artificial Sequence
  synthetic
  Xaa (1)..(1): Xaa is pyroglutamate
XGPPGQWSQVC

SEQ ID NO: 69 - protein, 11 aa, Artificial Sequence
  synthetic
  Xaa (1)..(1): Xaa is pyroglutamate
XGPPLQWMQAC

SEQ ID NO: 70 - protein, 11 aa, Artificial Sequence
  synthetic
  Xaa (1)..(1): Xaa is pyroglutamate
XGPPLQWFQVC

SEQ ID NO: 71 - protein, 11 aa, Artificial Sequence
  synthetic
  Xaa (1)..(1): Xaa is pyroglutamate
XGPPLQWTQVC

SEQ ID NO: 72 - protein, 11 aa, Artificial Sequence
  synthetic
  Xaa (1)..(1): Xaa is pyroglutamate
XGPPLSWLQVC

SEQ ID NO: 73 - protein, 11 aa, Artificial Sequence
  synthetic
  Xaa (1)..(1): Xaa is pyroglutamate
XGPLSQASQVC

SEQ ID NO: 74 - protein, 11 aa, Artificial Sequence
  synthetic
  Xaa (1)..(1): Xaa is pyroglutamate
XGPLSQAFQVC

SEQ ID NO: 75 - protein, 11 aa, Artificial Sequence
  synthetic
  Xaa (1)..(1): Xaa is pyroglutamate
XGPLSQSSQVC

SEQ ID NO: 76 - protein, 11 aa, Artificial Sequence
  synthetic
  Xaa (1)..(1): Xaa is pyroglutamate
XGPLSSQSQVC

SEQ ID NO: 77 - protein, 11 aa, Artificial Sequence
  synthetic
  Xaa (1)..(1): Xaa is pyroglutamate
XGPLSGWAQVC

SEQ ID NO: 78 - protein, 11 aa, Artificial Sequence
  synthetic
  Xaa (1)..(1): Xaa is pyroglutamate
XGPLSQWQQVC

SEQ ID NO: 79 - protein, 11 aa, Artificial Sequence
  synthetic
  Xaa (1)..(1): Xaa is pyroglutamate
XGPYSSDTTPC

SEQ ID NO: 80 - protein, 11 aa, Artificial Sequence
  synthetic
MSPPLSDTTPC

SEQ ID NO: 81 - protein, 11 aa, Artificial Sequence
  synthetic
MSPYSMQTTPC

SEQ ID NO: 82 - protein, 11 aa, Artificial Sequence
  synthetic
MSPLSSWLQVC

SEQ ID NO: 83 - protein, 11 aa, Artificial Sequence
  synthetic
MSPLSSQAQVC

SEQ ID NO: 84 - protein, 11 aa, Artificial Sequence
  synthetic
  Xaa (1)..(1): Xaa is pyroglutamate
XGPLSGWLQVC

SEQ ID NO: 85 - protein, 11 aa, Artificial Sequence
  synthetic
  Xaa (1)..(1): Xaa is pyroglutamate
XGPLSGQSQVC

SEQ ID NO: 86 - protein, 11 aa, Artificial Sequence
  synthetic
  Xaa (1)..(1): Xaa is pyroglutamate
XGPPGDWLQVC

SEQ ID NO: 87 - protein, 11 aa, Artificial Sequence
  synthetic
  Xaa (1)..(1): Xaa is pyroglutamate
XGPPLMSVLAC

SEQ ID NO: 88 - protein, 11 aa, Artificial Sequence
  synthetic
  Xaa (1)..(1): Xaa is pyroglutamate
XGPPLMGLQVC

SEQ ID NO: 89 - protein, 11 aa, Artificial Sequence
  synthetic
  Xaa (1)..(1): Xaa is pyroglutamate
XGPPLMALQVC

SEQ ID NO: 90 - protein, 11 aa, Artificial Sequence
  synthetic
  Xaa (1)..(1): Xaa is pyroglutamate
XGPPLMRLQVC

SEQ ID NO: 91 - protein, 11 aa, Artificial Sequence
  synthetic
  Xaa (1)..(1): Xaa is pyroglutamate
XGPPLMTLQVC

SEQ ID NO: 92 - protein, 11 aa, Artificial Sequence
  synthetic
  Xaa (1)..(1): Xaa is pyroglutamate
XGPPLMVTQSC

SEQ ID NO: 93 - protein, 11 aa, Artificial Sequence
  synthetic
  Xaa (1)..(1): Xaa is pyroglutamate
XGPPLMSLQSC

SEQ ID NO: 94 - protein, 11 aa, Artificial Sequence
  synthetic
  Xaa (1)..(1): Xaa is pyroglutamate
XGPPLMSGQSC

SEQ ID NO: 95 - protein, 11 aa, Artificial Sequence
  synthetic
  Xaa (1)..(1): Xaa is pyroglutamate
XGPPLMSSQSC

SEQ ID NO: 96 - protein, 11 aa, Artificial Sequence
  synthetic
  Xaa (1)..(1): Xaa is pyroglutamate
XGPPLMSLTVC

SEQ ID NO: 97 - protein, 7 aa, Artificial Sequence
  synthetic
  Xaa (1)..(1): Xaa is pyroglutamate
XGALRQC

SEQ ID NO: 98 - protein, 7 aa, Artificial Sequence
  synthetic
  Xaa (1)..(1): Xaa is pyroglutamate
XGVARNC

SEQ ID NO: 99 - protein, 7 aa, Artificial Sequence
  synthetic
  Xaa (1)..(1): Xaa is pyroglutamate
XGVARRC

SEQ ID NO: 100 - protein, 7 aa, Artificial Sequence
  synthetic
  Xaa (1)..(1): Xaa is pyroglutamate
XGVQRIC

SEQ ID NO: 101 - protein, 7 aa, Artificial Sequence
  synthetic
YQASEDC

SEQ ID NO: 102 - protein, 7 aa, Artificial Sequence
  synthetic
YQSREDC

SEQ ID NO: 103 - protein, 7 aa, Artificial Sequence
  synthetic
YSQREDC

SEQ ID NO: 104 - protein, 8 aa, Artificial Sequence
  synthetic
  Xaa (1)..(1): Xaa is pyroglutamate
XGAFQPDC

SEQ ID NO: 105 - protein, 8 aa, Artificial Sequence
  synthetic
  Xaa (1)..(1): Xaa is pyroglutamate
XGGFKQDC

SEQ ID NO: 106 - protein, 8 aa, Artificial Sequence
  synthetic
  Xaa (1)..(1): Xaa is pyroglutamate
XGFLTADC

SEQ ID NO: 107 - protein, 8 aa, Artificial Sequence
  synthetic
  Xaa (1)..(1): Xaa is pyroglutamate
XGLLQQDC

SEQ ID NO: 108 - protein, 8 aa, Artificial Sequence
  synthetic
KDLQFEDC

SEQ ID NO: 109 - protein, 8 aa, Artificial Sequence
  synthetic
LDAQFEDC

SEQ ID NO: 110 - protein, 8 aa, Artificial Sequence
  synthetic
TDIQFEDC

SEQ ID NO: 111 - protein, 8 aa, Artificial Sequence
  synthetic
VDGQFEDC

SEQ ID NO: 112 - protein, 8 aa, Artificial Sequence
  synthetic
EFLRFEDC

SEQ ID NO: 113 - protein, 8 aa, Artificial Sequence
  synthetic
GQLKFEDC

SEQ ID NO: 114 - protein, 8 aa, Artificial Sequence
  synthetic
ITQRFEDC

SEQ ID NO: 115 - protein, 8 aa, Artificial Sequence
  synthetic
SIQRFEDC

SEQ ID NO: 116 - protein, 8 aa, Artificial Sequence
  synthetic
  Xaa (1)..(1): Xaa is pyroglutamate
XGIQFIDC

SEQ ID NO: 117 - protein, 8 aa, Artificial Sequence
  synthetic
  Xaa (1)..(1): Xaa is pyroglutamate
XGIWIIDC

SEQ ID NO: 118 - protein, 8 aa, Artificial Sequence
  synthetic
  Xaa (1)..(1): Xaa is pyroglutamate
XGIWQYDC

SEQ ID NO: 119 - protein, 8 aa, Artificial Sequence
  synthetic
  Xaa (1)..(1): Xaa is pyroglutamate
XGVQYGDC

SEQ ID NO: 120 - protein, 8 aa, Artificial Sequence
  synthetic
  Xaa (1)..(1): Xaa is pyroglutamate
XLLWFEDC

SEQ ID NO: 121 - protein, 8 aa, Artificial Sequence
  synthetic
  Xaa (1)..(1): Xaa is pyroglutamate
XGDIQPDC

SEQ ID NO: 122 - protein, 8 aa, Artificial Sequence
  synthetic
  Xaa (1)..(1): Xaa is pyroglutamate
XGDQPIDC

SEQ ID NO: 123 - protein, 7 aa, Artificial Sequence
  synthetic
RRAEEDC

SEQ ID NO: 124 - protein, 7 aa, Artificial Sequence
  synthetic
RRKQEDC

SEQ ID NO: 125 - protein, 7 aa, Artificial Sequence
  synthetic
  Xaa (1)..(1): Xaa is pyroglutamate
XGKSQGC

SEQ ID NO: 126 - protein, 7 aa, Artificial Sequence
  synthetic
  Xaa (1)..(1): Xaa is pyroglutamate
XGRQAQC

SEQ ID NO: 127 - protein, 7 aa, Artificial Sequence
  synthetic
  Xaa (1)..(1): Xaa is pyroglutamate
XGRSQQC

SEQ ID NO: 128 - protein, 7 aa, Artificial Sequence
  synthetic
  Xaa (1)..(1): Xaa is pyroglutamate
XSKREDC

SEQ ID NO: 129 - protein, 7 aa, Artificial Sequence
  synthetic
  Xaa (1)..(1): Xaa is pyroglutamate
XYKQEDC

SEQ ID NO: 130 - protein, 7 aa, Artificial Sequence
  synthetic
  Xaa (1)..(1): Xaa is pyroglutamate
XGAWWRC

SEQ ID NO: 131 - protein, 7 aa, Artificial Sequence
  synthetic
  Xaa (1)..(1): Xaa is pyroglutamate
XGELHQC

SEQ ID NO: 132 - protein, 7 aa, Artificial Sequence
  synthetic
  Xaa (1)..(1): Xaa is pyroglutamate
XGQVWLC

SEQ ID NO: 133 - protein, 7 aa, Artificial Sequence
  synthetic
  Xaa (1)..(1): Xaa is pyroglutamate
XGQWSGC

SEQ ID NO: 134 - protein, 8 aa, Artificial Sequence
  synthetic
  Xaa (1)..(1): Xaa is pyroglutamate
XGQYLDDC

SEQ ID NO: 135 - protein, 8 aa, Artificial Sequence
  synthetic
  Xaa (1)..(1): Xaa is pyroglutamate
XGSQLQDC

SEQ ID NO: 136 - protein, 8 aa, Artificial Sequence
  synthetic
GRDQFEDC

SEQ ID NO: 137 - protein, 8 aa, Artificial Sequence
  synthetic
GREQFEDC

SEQ ID NO: 138 - protein, 7 aa, Artificial Sequence
  synthetic
VQRLEDC

SEQ ID NO: 139 - protein, 7 aa, Artificial Sequence
  synthetic
QGVFEDC

SEQ ID NO: 140 - protein, 7 aa, Artificial Sequence
  synthetic
AGVFEDC

SEQ ID NO: 141 - protein, 7 aa, Artificial Sequence
  synthetic
QAVFEDC

SEQ ID NO: 142 - protein, 7 aa, Artificial Sequence
  synthetic
QDAFEDC

SEQ ID NO: 143 - protein, 7 aa, Artificial Sequence
  synthetic
QGVAEDC

SEQ ID NO: 144 - protein, 7 aa, Artificial Sequence
  synthetic
QGVFADC

SEQ ID NO: 145 - protein, 7 aa, Artificial Sequence
  synthetic
QGVFEAC

SEQ ID NO: 146 - protein, 8 aa, Artificial Sequence
  synthetic
QGAVFEDC

SEQ ID NO: 147 - protein, 8 aa, Artificial Sequence
  synthetic
QGVFAEDC

SEQ ID NO: 148 - protein, 8 aa, Artificial Sequence
  synthetic
QGVFEDAC

SEQ ID NO: 149 - protein, 9 aa, Artificial Sequence
  synthetic
QGVFEDASC